SEQUENCE LISTING (35 sequences)

SEQ ID NO: 1 - protein, 9 aa, Human immunodeficiency virus type 1
RKKRRQRRR

SEQ ID NO: 2 - protein, 13 aa, Human immunodeficiency virus type 1
GRKKRRQRRRTPQ

SEQ ID NO: 3 - protein, 11 aa, Human immunodeficiency virus type 1
YGRKKRRQRRR

SEQ ID NO: 4 - protein, 26 aa, Artificial Sequence
  Description of Artificial Sequence Synthetic Kaposi FGF construct
AAVALLPAVLLALLAPVQRKRQKLMP

SEQ ID NO: 5 - protein, 17 aa, Caiman crocodilus
MGLGLHLLVLAAALQGA

SEQ ID NO: 6 - protein, 23 aa, Artificial Sequence
  Description of Artificial Sequence Synthetic HIV envelope glycoprotein gp41
GALFLGFLGAAGSTMGAPKSKRK

SEQ ID NO: 7 - protein, 16 aa, Drosophila sp.
RQIKIWFQNRRMKWKK

SEQ ID NO: 8 - protein, 5 aa, Artificial Sequence
  Description of Artificial Sequence Synthetic RGD peptide
XRGDX

SEQ ID NO: 9 - protein, 24 aa, Influenza virus
GLFEAIAGFIENGWEGMIDGGGYC

SEQ ID NO: 10 - protein, 27 aa, Unknown Organism
  Description of Unknown Organism Transportan A
GWTLNSAGYLLGKINLKALAALAKKIL

SEQ ID NO: 11 - protein, 9 aa, Unknown Organism
  Description of Unknown Organism Pre-S-peptide
SDHQLNPAF

SEQ ID NO: 12 - protein, 9 aa, Unknown Organism
  Description of Unknown Organism Somatostatin (tyr-3-octreotate)
SFCYWKTCT

SEQ ID NO: 13 - DNA, 21 bp, Artificial Sequence
  Description of Combined DNA/RNA Molecule Synthetic Fas sequence
gugcaagugcaaaccagactt

SEQ ID NO: 14 - DNA, 21 bp, Artificial Sequence
  Description of Combined DNA/RNA Molecule Synthetic Fas sequence
gucugguuugcacuugcactt

SEQ ID NO: 15 - DNA, 19 bp, Homo sapiens
gaggaagactgttactaca

SEQ ID NO: 16 - DNA, 19 bp, Homo sapiens
tgatgaaggacatggctta

SEQ ID NO: 17 - DNA, 19 bp, Homo sapiens
gaagcgtatgacacattga

SEQ ID NO: 18 - DNA, 19 bp, Homo sapiens
ggacattactagtgactca

SEQ ID NO: 19 - DNA, 1008 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 20 - DNA, 945 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaagtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 21 - DNA, 663 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctatgttgacttga

SEQ ID NO: 22 - DNA, 450 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatgatgtgaacatggaatcatcaaggaatgcacactcaccagcaacaccaagtgcaaagaggaaggatccagatctaacttggggtggctttgtcttcttcttttgccaattccactaa

SEQ ID NO: 23 - DNA, 399 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatgatgtgaacatggaatcatcaaggaatgcacactcaccagcaacaccaagtgcaaagaggaagtga

SEQ ID NO: 24 - DNA, 261 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccagatgtgaacatggaatcatcaaggaatgcacactcaccagcaacaccaagtgcaaagaggaagtga

SEQ ID NO: 25 - DNA, 312 bp, Homo sapiens
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccagatgtgaacatggaatcatcaaggaatgcacactcaccagcaacaccaagtgcaaagaggaaggatccagatctaacttggggtggctttgtcttcttcttttgccaattccactaa

SEQ ID NO: 26 - DNA, 450 bp, Homo sapiens; identical to SEQ ID NO: 22

SEQ ID NO: 27 - DNA, 19 bp, Mus musculus
gtgcaagtgcaaaccagac

SEQ ID NO: 28 - DNA, 19 bp, Homo sapiens
gtgcagatgtaaaccaaac

SEQ ID NO: 29 - DNA, 19 bp, Mus musculus
atacatcccgagaattgct

SEQ ID NO: 30 - DNA, 19 bp, Homo sapiens
atatatcaccactattgct

SEQ ID NO: 31 - DNA, 19 bp, Mus musculus
aagccgaatgtcgcagaac

SEQ ID NO: 32 - DNA, 19 bp, Homo sapiens
aagccaatctttgtactct

SEQ ID NO: 33 - RNA, 21 nt, Artificial Sequence
  Description of Artificial Sequence Synthetic GFP sequence
ggcuacguccaggagcgcacc

SEQ ID NO: 34 - RNA, 21 nt, Artificial Sequence
  Description of Artificial Sequence Synthetic GFP sequence
ugcgcuccuggacguagccuu

SEQ ID NO: 35 - DNA, 23 bp, Artificial Sequence
  Description of Artificial Sequence Synthetic DNA motif
aannnnnnnnnnnnnnnnnnntt

(19) United States
(12) Patent Application Publication
(10) Pub. No.: US 2008/0227733 A1
(43) Pub. Date: Sep. 18, 2008

(54) Method for Treating and Preventing Ischemia-Reperfusion Injury Using Rna Interfering Agent

(75) Inventors: Judy Lieberman, Brookline, MA (US); Peter Hamar, Budapest (HU); Erwei Song, Guang Dong (CN)

Correspondence Address: DAVID S. RESNICK, NIXON PEABODY LLP, 100 SUMMER STREET, BOSTON, MA, 02110-2131, US

(73) Assignee: Immune Disease Institute, Inc., Boston, MA (US)

(21) Appl. No.: 10/577,814

(22) PCT Filed: Nov. 1, 2004
(86) PCT No.: PCT/US2004/036200

§ 371 Date: Jan. 12, 2007

Related U.S. Application Data
(60) Provisional application No. 60/516,172, filed on Oct. 30, 2003.

(51) Int. Cl. A61K 31/7052; C12N 15/00; A01N 1/02; A61P 9/10; C12N 15/87

(52) U.S. Cl. 514/44; 435/440; 435/455; 435/1.1

(57) ABSTRACT

The present invention is based, at least in part, on the discovery of methods useful in the modulation, e.g., inhibition, of gene expression or protein activity, e.g., apoptosis-related gene expression, e.g., Fas gene expression or cytokine expression, e.g., proinflammatory cytokine expression. In particular, the present invention is based on novel RNA interfering agents, e.g., siRNA in reduction, e.g., prolonged reduction, of apoptosis-related gene expression or cytokine expression in cells. Inhibition of apoptosis-related gene expression or protein activity or cytokine gene expression or protein activity, e.g. by the siRNAs used in the methods of the invention, inhibits ischemia-reperfusion injury.

### CROSS-REFERENCE TO RELATED APPLICATIONS

The present application claims priority under 35 U.S.C. 119(e) to U.S. provisional application No. 60/516,172, filed Oct. 30, 2003, and which is herein incorporated by reference in its entirety.

### BACKGROUND OF THE INVENTION

The reduction in transport of blood, oxygen, and nutrients through the blood vessels of an organism can result in ischemia, necrosis, organ failure, and ultimately death of the organism. Unfortunately, reperfusion, although it relieves or reduces the problems caused by ischemia, is often followed by morphological and functional changes that ultimately result in tissue damage known as reperfusion injury, which significantly reduces the benefit of reperfusion. Reperfusion injury can be caused by either an acceleration of processes initiated during ischemia per se, or new pathophysiological changes that are initiated by the reperfusion itself leading to what is referred to as ischemia-reperfusion injury.

Apoptosis is believed to play a role in ischemia-induced cell death (Paschen, W (2003) J. Cereb. Blood Flow Metab. 23(7):773-9). Northern blot hybridization of mouse tissues have indicated that Fas (CD95) mRNA is abundantly expressed in the thymus, liver, heart, lung, kidney and ovary, but is weakly expressed in various other tissues (Maruyama, H., et al. (2002) Hum. Gene. Ther. 13: 455-68). Endothelial cells are targets of injury in the early cytotoxic phase of reperfusion. Initial cytotoxic cells are a source of reactive oxygen species (ROS) and proinflammatory mediators, such as tumor necrosis factor (TNF)-alpha with subsequent neutrophil activation and recruitment (Teoh, N.C. and Farrell, G. C. (2003) J. Gastroenterol. Hepatol. 18(8):891-902). Recruited neutrophils produce more ROS, which aggravates injury by oxidation of lipids and oxidative DNA damage (Reiter, R. J., et al. (2003) Ann. N.Y. Acad. Sci. 993:3547; Floyd, R. A., et al. (1992) Ann. Neurol. 32:S22-S27; DelZoppo, G. J. (1997) Repefusion damage: the role of PMN leucocytes. In Primer in Cerebrovascular Diseases. K. M. A. Welch, L. R. Caplan, D. J. Reis, et al, Eds.: 217-220. Academic Press, San Diego). Apoptosis has been implicated to be responsible for cell death during reperfusion, and this secondary cell death accounts for most of the lost parenchymal volume.

### SUMMARY OF THE INVENTION

The present invention is based, at least in part, on the discovery of methods useful in the modulation, e.g., inhibition, of ischemia-reperfusion injury. In particular, the present invention is based on RNA interfering agents, e.g., small interfering RNA (siRNA) molecules which target Fas-related genes, e.g., Fas pathway molecules, e.g., Fas or FasL, or cytokines, e.g., proinflammatory cytokines, e.g., IL-1 or TNFα, and result in reduction, e.g., prolonged reduction, of apoptosis-related gene expression, e.g., Fas pathway molecule, e.g., Fas or FasL, or cytokine, e.g., proinflammatory cytokine, e.g., IL-1 or TNFα gene expression, in cells, e.g., endothelial or epithelial cells, e.g., tubular cells or cardiac cells. In yet another embodiment, the RNA interfering agents of the invention may be administered to a subject to treat, e.g., therapeutically or prophylactically, an ischemia-reperfusion injury, in, e.g., kidney, heart, brain, liver, gut or lung tissue.

Accordingly, in one embodiment, the present invention provides a method for preventing ischemia reperfusion injury in an organ the method comprising the steps of administering to the organ in an individual in need thereof, a small interfering RNA (siRNA) directed against Fas mRNA in the amount which is capable of inhibiting the translation of Fas in the cells of the organ thereby preventing ischemia.

The individual in need of refers to an individual at risk of developing ischemia reperfusion injury, such as an organ transplant recipient, a person suspected of having ischemia reperfusion injury or a person having ischemia reperfusion injury.

In another embodiment, the invention provides a method of treating ischemia reperfusion injury in an organ the method comprising the steps of administering to the organ in an individual in need thereof, a siRNA directed against Fas mRNA in the amount which is capable of inhibiting the translation of Fas in the cells of the organ thereby treating ischemia. The term “treating” refers to either reversing the Fas-mediated cell death or reducing the Fas-mediated cell death in the target organ.

In one embodiment, the siRNAs are selected from the human Fas (hFas) sequences, wherein the siRNAs are preferably 21-23 bp in length.

In one preferred embodiment, the siRNAs are selected from the group consisting of hFas siRNA 1 (beginning at nucleotide 457) 5′-GAGGAAGACTGTTACTACA-3′, hFas siRNA 2 (beginning at nucleotide 667) 5′-TGATGAAGGACATGGCTTA-3′, hFas siRNA 3 (beginning at nucleotide 1211) 5′-GAAGCGTATGACACATTGA-3′, and hFas siRNA 4 (beginning at nucleotide 1294) 5′-GGACATTACTAGTGACTCA-3′.

In one preferred embodiment, the organ is selected from kidney, liver, lung, and heart. In a more preferred embodiment, the organ is kidney or liver.

In one embodiment, the individual in need of prevention of ischemia reperfusion injury is an organ transplant donor or and organ transplant recipient. In a more preferred embodiment, the organ transplant donor or recipient is a kidney or liver transplant donor or recipient.

In one preferred embodiment, the siRNA is delivered into the one or more blood supply vessels of the organ. In a more preferred embodiment, the siRNA is delivered to renal vein, if the treatment is to prevent ischemia reperfusion injury in kidney or hepatic vein if the treatment is to prevent ischemia reperfusion injury in liver. The delivery is preferably via catheterization of the blood supply vessel, such as the renal vein or the hepatic vein.

The siRNA may be chemically modified using modifications suitable for oligonucleotide modification in the antisense methodology. One preferred siRNA modification is an siRNA duplex containing either phosphodiester or one or more phosphothioate linkages. Other preferred modifications include 2′-deoxy-2′-fluorouridine and locked nucleic acid (LNA) nucleotides. Preferably, the modifications involve minimal 2′-O-methyl modification, preferably excluding such modification. The modifications also preferably exclude modifications of the free 5′-hydroxyl groups of the siRNA.

In a preferred embodiment, the siRNA or modified siRNA is delivered to the organ in a pharmaceutically acceptable carrier. Additional carrier agents, such as liposomes, may be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, Fas. In one embodiment, the vector may be a regulatable vector, such as tetracycline inducible vector.

In one preferred embodiment, the siRNA is delivered using the siRNA delivery system described in U.S. provisional application No. 60/601,950 filed Aug. 16, 2004.

### BRIEF DESCRIPTION OF DRAWINGS

FIGS. 1A-1G show that a single hydrodynamic injection of Fas siRNA silences Fas expression in kidneys subjected to 35 min of ischemia.

FIGS. 2A-2E show Fas silencing after hydrodynamic and renal vein injection of Fas siRNA.

FIGS. 3A-3C show that hydrodynamic or renal vein injection of Fas siRNA protects mice from lethal kidney ischemia: survival and BUN levels of surviving mice after 35 min of kidney ischemia and reperfusion.

### DETAILED DESCRIPTION OF THE INVENTION

The present invention is based, at least in part, on the discovery of methods useful in the modulation, e.g., inhibition, of ischemia-reperfusion injury to cells, tissues, and organs. In particular, the present invention is based on RNA interfering agents, e.g., small interfering RNA (siRNA) molecules which target apoptosis-related genes, e.g., Fas-related genes, e.g., Fas pathway molecules, e.g. Fas or FasL, or cytokines, e.g., proinflammatory cytokines, e.g., IL-1 or TNFα, and result in reduction, of apoptosis-related gene expression, e.g., Fas pathway molecule, e.g., Fas or FasL, or cytokine, e.g., proinflammatory cytokine, e.g., IL-1 or TNFα gene expression, in cells, e.g., endothelial or epithelial cells, e.g., tubular cells of the kidney or cardiac cells. It has been shown that ischemia-reperfusion injury and mortality is inhibited by administration of an RNA interfering agent, e.g., an siRNA, targeting an apoptosis related gene, e.g., Fas, via intravenous injection.

Accordingly, in one embodiment, the invention provides a method of administration of an RNA interfering agent which targets Fas, preferably human Fas (hFas).

The hFas protein encoded by hFas gene is a member of the TNF-receptor superfamily. This receptor contains a death domain. It has been shown to play a central role in the physiological regulation of programmed cell death, and has been implicated in the pathogenesis of various malignancies and diseases of the immune system. The interaction of this receptor with its ligand allows the formation of a death-inducing signaling complex that includes Fas-associated death domain protein (FADD), caspase 8, and caspase 10. The autoproteolytic processing of the caspases in the complex triggers a downstream caspase cascade, and leads to apoptosis. Fas receptor has been also shown to activate NF-kappaB, MAPK3/ERK1, and MAPK8/JNK, and is found to be involved in transducing the proliferating signals in normal diploid fibroblast and T cells. At least eight alternatively spliced transcript variants encoding seven distinct isoforms have been described. The isoforms lacking the transmembrane domain may negatively regulate the apoptosis mediated by the full length isoform. Therefore, the preferred target for the inhibition of hFas are the regions in the full length isoform of Fas. The most preferred siRNA molecules include: hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3 [SEQ ID NO: 17], and hFas siRNA 4 (beginning at nucleotide 1294) 5′-GGACATTACTAGTGACTCA-3′ [SEQ ID NO: 18].

Accordingly, the RNA interfering molecules can be designed to target sequences including, but not limited to 1. NM_{—}000043 (cDNA:1008 nt), Homo sapiens tumor necrosis factor receptor superfamily, member 6 (TNFRSF6), transcript variant 1, mRNA, this variant (1) encodes the longest isoform (1); NM_{—}152871 (cDNA:945 nt), Homo sapiens tumor necrosis factor receptor superfamily, member 6 (TNFRSF6), transcript variant 2, mRNA, this variant (2) lacks an in-frame coding segment compared to variant 1, resulting an isoform (2) that lacks an internal region, as compared to isoform 1; NM_{—}152872 (cDNA:663 nt), Homo sapiens tumor necrosis factor receptor superfamily, member 6 (TNFRSF6), transcript variant 3, mRNA, this variant (3) lacks a coding segment, which leads to a translation frameshift, compared to variant 1. The resulting isoform (3) contains a distinct and shorter C-terminus, as compared to isoform 1; NM_{—}152873 (cDNA:450 nt); Homo sapiens tumor necrosis factor receptor superfamily, member 6 (TNFRSF6), transcript variant 4, mRNA, this variant (4) lacks a coding segment, which leads to a translation frameshift, compared to variant 1 and the resulting isoform (4) contains a distinct and shorter C-terminus, as compared to isoform 1; NM_{—}152875 (cDNA:399 nt), Homo sapiens tumor necrosis factor receptor superfamily, member 6 (TNFRSF6), transcript variant 5, mRNA, this variant (5) lacks two coding segments, which leads to a translation frameshift, compared to variant 1, the resulting isoform (5) contains a distinct and shorter C-terminus, as compared to isoform 1; NM_{—}152876 (cDNA:261 nt), Homo sapiens tumor necrosis factor receptor superfamily, member 6 (TNFRSF6), transcript variant 6, mRNA, this variant (6) lacks two coding segments, which leads to a translation frameshift, compared to variant 1, the resulting isoform (6) contains a distinct and shorter C terminus, as compared to isoform 1; NM_{—}152877 (cDNA:312 nt), Homo sapiens tumor necrosis factor receptor superfamily, member 6 (TNFRSF6), transcript variant 7, mRNA, this variant (7) lacks a coding segment, which leads to a translation frameshift, compared to variant 1 and the resulting isoform (7) contains a distinct and shorter C-terminus, as compared to isoform 1 and NM_{—}152874 (cDNA:450 nt), Homo sapiens tumor necrosis factor receptor superfamily, member 6 (TNFRSF6), transcript variant 8, mRNA, this variant (8) lacks two coding segments, which leads to a translation frameshift, compared to variant 1. The resulting isoform (4) contains a distinct and shorter C-terminus, as compared to isoform 1.

The accession numbers refer to NCBI database.

The complete cDNA sequences of the different TNFRSF6 variants are as follows:

Human FAS (hFAS) variant#1:

[SEQ ID NO: 19]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGGATCCAGATCTAACTTGGGGTGGCTTTGTCTTCTTCTTTTGCCAA
TTCCACTAATTGTTTGGGTGAAGAGAAAGGAAGTACAGAAAACATGCAGA
AAGCACAGAAAGGAAAACCAAGGTTCTCATGAATCTCCAACCTTAAATCC
TGAAACAGTGGCAATAAATTTATCTGATGTTGACTTGAGTAAATATATCA
CCACTATTGCTGGAGTCATGACACTAAGTCAAGTTAAAGGCTTTGTTCGA
AAGAATGGTGTCAATGAAGCCAAAATAGATGAGATCAAGAATGACAATGT
CCAAGACACAGCAGAACAGAAAGTTCAACTGCTTCGTAATTGGCATCAAC
TTCATGGAAAGAAAGAAGCGTATGACACATTGATTAAAGATCTCAAAAAA
GCCAATCTTTGTACTCTTGCAGAGAAAATTCAGACTATCATCCTCAAGGA
CATTACTAGTGACTCAGAAAATTCAAACTTCAGAAATGAAATCCAAAGCT
TGGTCTAG
Human FAS (hFAS) variant#2:

[SEQ ID NO: 20]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCAGAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGGCTTAGAAGTGGAAAT
AAACTGCACCCGGACCCAGAATACCAAGTGCAGATGTAAACCAAACTTTT
TTTGTAACTCTACTGTATGTGAACACTGTGACCCTTGCACCAAATGTGAA
CATGGAATCATCAAGGAATGCACACTCACCAGCAACACCAAGTGCAAAGA
GGAAGTGAAGAGAAAGGAAGTACAGAAAACATGCAGAAAGCACAGAAAGG
AAAAGCAAGGTTCTCATGAATCTCCAACCTTAAATCCTGAAACAGTGGCA
ATAAATTTATCTGATGTTGACTTGAGTAAATATATCACCACTATTGCTGG
AGTCATGACACTAAGTCAAGTTAAAGGCTTTGTTCGAAAGAATGGTGTCA
ATGAAGCCAAAATAGATGAGATCAAGAATGACAATGTCCAAGACACAGCA
GAACAGAAAGTTCAACTGCTTCGTAATTGGCATCAACTTCATGGAAAGAA
AGAAGCGTATGACACATTGATTAAAGATCTCAAAAAAGCCAATCTTTGTA
CTCTTGCAGAGAAAATTCAGAGTATCATCCTCAAGGACATTACTAGTGAC
TCAGAAAATTCAAACTTCAGAAATGAAATGGAAAGCTTGGTCTAG
Human FAS (hFAS) variant#3:

[SEQ ID NO: 21]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGUGAGACTCAGAACTTGGAAG
GCCTGCATCATGATGGCCAATTCTGCCATAAGCCCTGTCCTCCAGGTGAA
AGGAAAGCTAGGGACTGCACAGTCAATGGGGATGAACCAGACTGCGTGCC
CTGCCAAGAAGGGAAGGAGTACACAGACAAAGCCCATTTTTCTTCCAAAT
GCAGAAGATGTAGATTGTGTGATGAAGGACATGGCTTAGAAGTGGAAATA
AACTGCACCCGGACCCAGAATACCAAGTGCAGATGTAAACCAAACTTTTT
TTGTAACTCTACTGTATGTGAACACTGTGACCCTTGCACCAAATGTGAAC
ATGGAATCATCAAGGAATGCACACTCACCAGCAACACCAAGTGCAAAGAG
GAAGGATCCAGATCTAACTTGGGGTGGCTTTGTCTTCTTCTTTTGCCAAT
TCCACTAATTGTTTGGGTGAAGAGAAAGGAAGTACAGAAAACATGCAGAA
AGCACAGAAAGGAAAACCAAGGTTCTCATGAATCTCCAACCTTAAATCCT
ATGTTGACTTGA
Human FAS (hFAS) variant#4:

[SEQ ID NO: 22]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTGTTCCAAA
TGCAGAAGATGTAGATTGTGTGATGAAGGACATGATGTGAACATGGAATC
ATCAAGGAATGCACACTCACCAGCAACACCAAGTGCAAAGAGGAAGGATC
CAGATCTACTTGGGGTGGCTTTGTCTTCTTCTTTTGCCAATTCCACTAA
Human FAS (hFAS) variant#5:

[SEQ ID NO: 23]

ATGCTGGGCATCTGGACCCTCCTACCTCTGGTTGTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGATGTGAACATGGAATC
ATGAAGGAATGCACACTCACCAGCAACACCAAGTGCAAAGAGGAAGTGA
Human FAS (hFAS) variant#6:

[SEQ ID NO: 24]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGAGTGTTACTACAGTTGAGACTCAGAACTTGGAA
GGCCTGCATCATGATGGCCAATTCTGCCATAAGCCCTGTCCTCCAGATGT
GAACATGGAATCATCAAGGAATGCACACTCACCAGCAACACCAAGTGCAA
AGAGGAAGTGA
Human FAS (hFAS) variant#7:

[SEQ ID NO: 25]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGATGT
GAACATGGAATCATCAAGGAATGCACACTCACCAGCAACACCAAGTGCAA
AGAGGAAGGATCCAGATCTAACTTGGGGTGGCTTTGTCTTCTTCTTTTGC
CAATTCCACTAA
Human FAS (hFAS) variant#8:

[SEQ ID NO: 26]

ATGCTGGGCATCTGGACCCTCCTACCTCTGGTTCTTACGTCTGTTGCTAG
ATTATCGTCCAAAAGTGTTAATGCCCAAGTGACTGACATCAACTCCAAGG
GATTGGAATTGAGGAAGACTGTTACTACAGTTGAGACTCAGAACTTGGAA
GGCCTGCATCATGATGGCCAATTCTGCCATAAGCCCTGTCCTCCAGGTGA
AAGGAAAGCTAGGGACTGCACAGTCAATGGGGATGAACCAGACTGCGTGC
CCTGCCAAGAAGGGAAGGAGTACACAGACAAAGCCCATTTTTCTTCCAAA
TGCAGAAGATGTAGATTGTGTGATGAAGGACATGATGTGAACATGGAATC
ATCAAGGAATGCACAGTCACCAGCAACACCAAGTGCAAAGAGGAAGGATG
CAGATCTAACTTGGGGTGGCTTTGTCTTCTTCTTTTGCCAATTCCACTAA

Gene alignment using the CLUSTAL X software of the human and mouse Fas sequences demonstrated that the target site of the Fas siRNAs that worked the most effectively in the mouse as described in the PCT application No. PCT/US03/34424, i.e., mouse siRNAs 1, 5 and 6, had 4, 5 and 10 mismatches, respectively. Due to the large number of mismatches and the placement of mismatches in regions that are known to be important for determining the specificity of silencing, new siRNAs that target the human Fas sequence were designed.

In one preferred embodiment, the siRNA target sites against the human Fas sequence are: hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3′ [SEQ ID NO: 17], and hFas siRNA 4 (beginning at nucleotide 1294) 5′-GGACATTACTAGTGACTCA-3′ [SEQ ID NO: 18].

These siRNAs were chosen to maximize the uptake of the antisense (guide) strand of the siRNA into RISC and thereby maximize the ability of RISC to target human Fas mRNA for degradation. This was accomplished by looking for sequences that had the lowest free energy of binding at the 5′-terminus of the antisense strand. The lower free energy would lead to an enhancement of the unwinding of the 5′-end of the antisense strand of the siRNA duplex, thereby ensuring that the antisense strand will be taken up by RISC and direct the sequence-specific cleavage of the human Fas mRNA.

Comparison of the mouse siRNAs target sequences with the human mRNA sequence

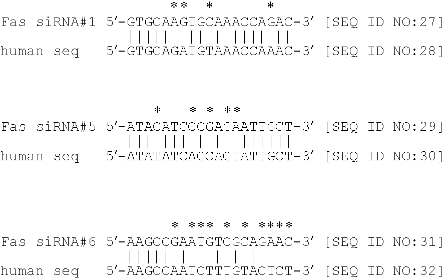

In addition, in one embodiment, the methods of the instant invention include administration of an RNA interfering agent which targets an apoptosis-related gene or a cytokine, e.g., a proinflammatory cytokine, to a subject to treat, e.g., therapeutically or prophylactically, an ischemia-reperfusion injury, e.g., due to stroke, heart attack, or any reduction in transport of blood, oxygen, and/or nutrients through the blood vessels of an organism causing ischemia followed by reperfusion of the cells and tissues. Therefore, in another embodiment, organ or tissue damage or acute organ failure due to cell death, e.g., kidney failure, due to reduced blood flow to the organ or tissue, may be prevented or treated using the methods of the invention.

The phrase “ischemia-reperfusion injury” refers to any injury or pathological changes to a cell, tissue, or organ that is related to or caused by ischemia or reperfusion. Ischemia refers to insufficient oxygen to a tissue or organ which may result in injury or pathological change to the affected cell, tissue, or organ caused by the insufficient blood flow and/or oxygen to the cell, tissue, or organ. Ischemia may result from any event leading to a decrease in bloodflow and/or oxygen to a tissue or organ, such as, for example, vascular disorders that result in occlusion of a vessel, including, for example stenosis, e.g., renal artery stenosis, myocardial infarction, thrombosis, or stroke, surgery, e.g., heart surgery or transplantation, e.g. the transplantation of allogeneic or xenogeneic tissue into a mammalian recipient. Ischemia may occur in any organ, tissue or cell type, including, for example, bone marrow, pancreas, stomach, cornea, kidney, lung, liver, heart, skin, brain, and spleen.

Reperfusion refers to the return of blood flow and oxygen to a cell, tissue, or organ, following ischemia. Reperfusion may lead to further injury or pathological changes to a cell, tissue or organ which may have been injured due to ischemia. Although the precise mechanism of reperfusion injury is uncertain, there is support for neutrophil-mediated cell injury as a contributing factor. Other possible mechanisms include platelet aggregation, vascular injury, local release of vasoactive substances, and depletion of the nucleotide pool.

Ischemia reperfusion injury may also occur during organ transplant. Accordingly, the present invention provides methods for prevention and/or treatment of ischemia reperfusion during the organ transfers. In one embodiment, the method comprises administering siRNA, preferably human Fas targeting siRNA, in a pharmaceutically acceptable carrier into the blood supply vein of the organ to be transplanted prior to or simultaneously with detaching the organ from the donor.

In one embodiment, the siRNA is administered at the time the organ is transplanted to the recipient or shortly thereafter, to the blood supply vein of the transplanted organ of the recipient.

In yet another embodiment, the siRNA is delivered to both the donor and the recipient.

In the preferred embodiment, the organ transplant is a human kidney or liver transplant and the blood supply vein is renal vein for kidney or hepatic vein for liver. The preferred delivery method is renal vein or hepatic vein catheterization.

Preferably the ischemia reperfusion injury as intended to be prevented or treated with the methods of the present invention is caused by Fas-related apoptosis. Apoptosis has been strongly implicated to be responsible for cell death during reperfusion. The importance of Fas mediated apoptosis in the pathology of ischemia-reperfusion has been demonstrated in a number of experimental settings. Without intending to be limited by theory, the primary injury during ischemia is likely necrosis due to oxygen deficiency and energy depletion. During reperfusion, a secondary injury may occur due to inflammation. Inflammatory infiltration by neutrophils and re-supplying oxygen results in oxidative stress, which induces apoptosis. Furthermore, it has been suggested that T-cells are involved in ischemia-reperfusion injury. These observations suggest an important role for apoptosis, e.g., Fas mediated apoptosis, in ischemia-reperfusion injury, e.g., ischemia-reperfusion injury of a cell, tissue or organ, including, but not limited to, kidney, heart, brain, liver, and lung tissue.

Target genes of RNA interfering agents used in the methods of the invention include apoptosis related genes. Preferably, the target gene is Fas, most preferably human Fas (hFas).

As used herein, an “apoptosis-related gene” or “apoptosis-related molecule”, also includes any upstream or downstream molecule that is involved in transducing or modulating an apoptotic signal, e.g., molecules involved in or related to apoptotic pathways known to the skilled artisan (see, e.g. Konopleva, M. et al. Drug Resistance in Leukemia and Lymphoma III, Chapter 24 (Kaspers et al. eds. 1999, incorporated herein by reference).

Apoptosis-related genes include, but are not limited to, Fas pathway molecules, e.g., Fas, FasL, and TNF-R1; caspases, e.g., Group I caspases, Group II caspases, and Group II caspases, flice, flip, fadd, and other pro-apoptotic genes as known in the art.

Fas pathway molecules include any molecule involved in or related to a pathway leading to apoptosis or programmed cell death induced by Fas. Fas pathway molecules include, but are not limited to Fas, the Fas ligand (FasL), and members of the TNFR superfamily of receptors. FADD, caspase 8, bid, and caspase 3 are also included as Fas pathway molecules.

The Fas pathway induces apoptosis by ligation of the Fas receptor on cells by FasL. The Fas receptor, also known as APO-1 or CD95, is a member of the TNFR superfamily of receptors. Other members of the TNFR family include TNF-R1, DR-3, DR-4 and DR-5, each with death domains that directly initiate apoptosis. Binding of FasL to the Fas receptor then leads to aggregation of the receptor on the cell membrane and specific recruitment of intracellular signaling molecules known as DISC, or death-inducing signal complex. The adaptor protein, FADD, binds to the intracellular death domain of Fas which leads to the recruitment of caspase-8, also known as FLICE or MACH. Fas-induced cell death may activate a pathway that alters mitochondrial permeability transition.

Ischemia-reperfusion injury initiates an inflammatory response which is believed to involve chemokines, e.g., proinflammatory chemokines, e.g., TNFalpha and other cytokines. Accordingly, cytokines are targets of the RNA interfering agents used in the methods of the invention. Cytokines include proinflammatory cytokines, e.g., IL-1β and TNFα, and anti-inflammatory cytokines, e.g., CSF2, CSF3, TGFβ.

Proinflammatory cytokine molecules include any immunoregulatory cytokine that accelerates or induces any aspect of inflammation due to, for example, injury, infection or any immunological disease or disorder or in response to apoptosis-related genes. A proinflammatory cytokine may act either as an endogenous pyrogen (e.g., IL1, TNFα), may upregulate the synthesis of secondary mediators and other pro-inflammatory cytokines by both macrophages and mesenchymal cells (including fibroblasts, epithelial and endothelial cells), may stimulate the production of acute phase proteins, or may attract inflammatory cells.

Proinflammatory cytokines include, but are not limited to, for example, ILα, IL1β, and TNFα, LIF, IFNγ, OSM, CNTF, TGFβ, GM-CSF, IL11, IL12, IL17, IL18, IL8, and a variety of other chemokines that chemoattract inflammatory cells.

Anti-inflammatory cytokine molecules include any immunoregulatory cytokine that counteracts any aspect of inflammation, e.g., cell activation or the production of pro-inflammatory cytokines, and thus contributes to the control of the magnitude of the inflammatory responses in vivo. In one embodiment, anti-inflammatory cytokines act by the inhibition of the production of pro-inflammatory cytokines or by counteracting many biological effects of pro-inflammatory mediators in different ways. Anti-inflammatory cytokines include, but are not limited to, for example, IL4, IL10, and IL13. Other anti-inflammatory mediators include IL16, IFNα, TGF, IL1ra, or G-CSF.

In one embodiment, the RNA interfering agents used in the methods of the invention, e.g., the siRNAs used in the methods of the invention, have been shown to be taken up actively by cells in vivo following intravenous injection, e.g., hydrodynamic injection, without the use of a vector, illustrating efficient in vivo delivery of the RNA interfering agents, e.g., the siRNAs used in the methods of the invention. Because silencing after duplex siRNA injection is prolonged but not permanent, long-term toxicity, such as lymphoproliferative or autoimmune disease, seen in humans with mutations of fas and in the lpr mouse (Takahashi, T. et al. (1994) Cell 76, 969-76), is of little concern.

One preferred method to deliver the siRNAs is catheterization of the blood supply vein of the target organ.

Other strategies for delivery of the RNA interfering agents, e.g. the siRNAs or shRNAs used in the methods of the invention, may also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

In one embodiment, the dsRNA, such as siRNA or shRNA, is delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the RNA interfering agents, e.g. the siRNAs used in the methods of the invention, can be introduced into cells, e.g., cultured cells, which are subsequently transplanted into the subject by, e.g., transplanting or grafting, or alternatively, can be obtained from a donor (i.e., a source other than the ultimate recipient), and applied to a recipient by, e.g., transplanting or grafting, subsequent to administration of the RNA interfering agents, e.g., the siRNAs of the invention, to the cells. Alternatively, the RNA interfering agents, e.g., the siRNAs of the invention, can be introduced directly into the subject in such a manner that they are directed to and taken up by the target cells and regulate or promote RNA interference of the target gene, e.g., apoptosis-related gene, e.g., Fas. The RNA interfering agents, e.g., the siRNAs of the invention, may be delivered singly, or in combination with other RNA interfering agents, e.g., siRNAs, such as, for example siRNAs directed to other cellular genes, e.g., other apoptosis-related genes. The RNA interfering agents, e.g., siRNAs of the invention may also be administered in combination with other pharmaceutical agents which are used to treat or prevent ischemia-reperfusion tissue or organ injury, e.g., liver, heart, brain, kidney, pancreas, stomach, spleen, lung. The preferred organs are kidney and liver.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi).

Preferably, the RNA interfering agent in the methods of the present invention is siRNA. The preferred siRNAs according to the present invention include Fas, preferably human Fas, targeting siRNAs. The human Fas targeting siRNAs are designed so as to maximize the uptake of the antisense (guide) strand of the siRNA into RNA-induced silencing complex (RISC) and thereby maximize the ability of RISC to target human Fas mRNA for degradation. This can be accomplished by looking for sequences that has the lowest free energy of binding at the 5′-terminus of the antisense strand. The lower free energy would lead to an enhancement of the unwinding of the 5′-end of the antisense strand of the siRNA duplex, thereby ensuring that the antisense strand will be taken up by RISC and direct the sequence-specific cleavage of the human Fas mRNA.

In one preferred embodiment, the human Fas targeting siRNA sequences are selected to from the group of siRNA target sequences consisting of: hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3′ [SEQ ID NO: 17], and hFas siRNA 4 (beginning at nucleotide 1294) 5′-GGACATTACTAGTGACTCA-3′ [SEQ ID NO: 18].

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

In one embodiment, the siRNA may target a specific genetic mutation in a target gene, such as human Fas. In another embodiment, the siRNA may target a sequence which is conserved between one or more target genes, such as different Fas variants discussed elsewhere in this description.

The target gene or sequence of the RNA interfering agent may be a cellular gene or genomic sequence. An siRNA may be substantially homologous to the target gene or genomic sequence, or a fragment thereof. As used herein, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target.

The siRNA preferably targets only one sequence. Each of the RNA interfering agents, such as siRNAs, can be screened for potential off-target effects may be analyzed using, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substitute for another sugar molecule or a molecule which performs a similar function. Moreover, a non-natural linkage between nucleotide residues may be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology.

Various aspects of the invention are described in further detail in the following subsections:

I. Short Interfering RNAs (siRNAs) of the Invention

In one embodiment, the preferred siRNA useful in the methods of treatment and/or prevention of ischemia reperfusion injury of the invention is an siRNA targeting Fas, preferably human Fas.

The preferred hFas-targeting sequences include, but are not limited to siRNAs targeting the sequences encoding human Fas sequence variants 1-8 [SEQ ID NOS: 19-26], described elsewhere in this description. The most preferred siRNA sequences useful according to the present invention include, but are not limited to: hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3′ [SEQ ID NO: 17], and hFas siRNA 4 (beginning at nucleotide 1294) 5′-GGACATTACTAGTGACTCA-3′ [SEQ ID NO: 18], or any combination comprising two or more of the SEQ ID NO:s 15-18.

Other siRNAs useful in treating ischemia reperfusion injury according to the methods of the present invention may be readily designed and tested. Accordingly, the present invention also relates to siRNA molecules of about 15 to about 40 or about 15 to about 28 nucleotides in length, which are homologous to an apoptosis-related gene, e.g., a Fas pathway molecule, e.g., Fas or FasL, or a cytokine, e.g., a proinflammatory cytokine, e.g., IL-1 or TNFα, and mediate RNAi of an apoptosis-related gene or a cytokine. Preferably, the siRNA molecules have a length of about 19 to about 25 nucleotides. More preferably, the siRNA molecules have a length of about 19, 20, 21, or 22 nucleotides. The siRNA molecules of the present invention can also comprise a 3′ hydroxyl group. The siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the RNA molecule is double stranded, one strand has a 3′ overhang and the other strand can be blunt-ended or have an overhang. In the embodiment in which the RNA molecule is double stranded and both strands comprise an overhang, the length of the overhangs may be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′hydroxyl significantly enhances the nuclease resistance of the overhang in tissue culture medium.

A. Design and Preparation of siRNA Molecules

Synthetic siRNA molecules, including shRNA molecules, of the present invention can be obtained using a number of techniques known to those of skill in the art. For example, the siRNA molecule can be chemically synthesized or recombinantly produced using methods known in the art, such as using appropriately protected ribonucleoside phosphomidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., an apoptosis-related gene or a cytokine, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (where N can be any nucleotide) and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

### Delivery of RNA Interfering Agents

Methods of delivering RNA interfering agents, e.g., an siRNA of the present invention, or vectors containing an RNA interfering agent, e.g., an siRNA of the present invention, to the target cells, e.g., tubular cells of the kidney, liver or cardiac cells, for uptake include injection of a composition containing the RNA interfering agent, e.g., an siRNA, or directly contacting the cell, e.g., a tubular cell of the kidney, liver or a cardiac cell, or tissue, e.g., heart, liver or kidney, with a composition comprising an RNA interfering agent, e.g., an siRNA. In another embodiment, RNA interfering agents, e.g., an siRNA may be injected directly into any blood vessel, such as vein, artery, venule or arteriole, via, e.g., hydrodynamic injection or catheterization. Administration may be by a single injection or by two or more injections. Preferably, the RNA interfering agent is delivered directly to the organ, such as kidney or liver, through blood vessels to such organs, such as renal vein or artery or hepatic vein or artery. The RNA interfering agent is delivered in a pharmaceutically acceptable carrier. One or more RNA interfering agents may be used simultaneously.

In one preferred embodiment, only one siRNA that targets human Fas is used. The delivery or administration of the siRNA is preferably performed in free form, i.e. without the use of vectors.

In another preferred embodiment, the delivery is performed using an siRNA delivery system described in U.S. provisional patent application No. 60/601,950 filed Aug. 16, 2004, and U.S. Patent Application Publication No. 20040023902, incorporated herein by reference in their entirety method of targeted delivery both in vitro and in vivo of small interference RNAs into desired cells thus avoiding entry of the siRNA into other than intended target cells. The method allows treatment of specific cells with RNA interference limiting potential side effects of RNA interference caused by non-specific targeting of RNA interference. The method used a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety may be either in the carboxyl-terminal or amino-terminal end of the construct or in the middle of the fusion protein.

A viral-mediated delivery mechanism may also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Other methods of introducing siRNA molecules of the present invention to target cells, e.g., tubular cells of the kidney or cardiac cells, include a variety of art-recognized techniques including, but not limited to, calcium phosphate or calcium chloride co-precipitation, DEAE-dextran-mediated transfection, lipofection, or electroporation as well as a number of commercially available transfection kits (e.g., OLIGOFECTAMINE® Reagent from Invitrogen) (see, e.g. Sui, G. et al. (2002) Proc. Natl. Acad. Sci. USA 99:5515-5520; Calegari, F. et al. (2002) Proc. Natl. Acad. Sci., USA Oct. 21, 2002 [electronic publication ahead of print]; J-M Jacque, K. Triques and M. Stevenson (2002) Nature 418:435-437; and Elbashir, S. M et al. (2001) supra). Suitable methods for transfecting a target cell, e.g., a tubular cell of the kidney or a cardiac cell can also be found in Sambrook, et al. (Molecular Cloning: A Laboratory Manual 2nd, ed., Cold Spring Harbor Laboratory, Cold Spring Harbor Laboratory Press, Cold Spring Harbor, N.Y., 1989), and other laboratory manuals. The efficiency of transfection may depend on a number of factors, including the cell type, the passage number, the confluency of the cells as well as the time and the manner of formation of siRNA- or shRNA-liposome complexes (e.g., inversion versus vortexing). These factors can be assessed and adjusted without undue experimentation by one with ordinary skill in the art.

The RNA interfering agents, e.g., the siRNAs or shRNAs of the invention, may be introduced along with components that perform one or more of the following activities: enhance uptake of the RNA interfering agents, e.g. siRNA, by the cell, e.g., tubular cells of the kidney or cardiac cells, inhibit annealing of single strands, stabilize single strands, or otherwise facilitate delivery to the target cell and increase inhibition of the target gene, e.g., FAS.

The RNA interfering agents, e.g., siRNA, may be directly introduced into the cell, e.g., a tubular cell of the kidney or cardiac cell, or introduced extracellularly into a cavity, interstitial space, into the circulation of an organism, introduced orally, or may be introduced by bathing a cell or organism in a solution containing the RNA interfering agent, e.g., an siRNA. RNA interfering agents, e.g. an siRNA, may also be introduced into cells via topical application to a mucosal membrane or dermally. Vascular or extravascular circulation, the blood or lymph system, and the cerebrospinal fluid are also sites where the agents may be introduced.

A further method of treating cells with siRNA is an ex vivo method wherein cells, e.g., tubular cells of the kidney or cardiac cells, to be treated with an RNA interfering agent, e.g., an siRNA, are obtained from the individual using known methods and one or more RNA interfering agents that mediate target gene expression are introduced into the cells, which are then re-introduced into the individual. In another embodiment, the cells, e.g., tubular cells of the kidney or cardiac cells, can be obtained from a donor (i.e., a source other than the ultimate recipient), modified by administering the RNA interfering agent(s), and applied to a recipient, again by transplanting or grafting.

For example, cells, e.g., tubular cells of the kidney, may be obtained from an individual or donor by, generally, removing all or a portion of an organ, e.g., a kidney, from which cells, e.g., tubular cells, are removed by in situ perfusion of a collagenase solution. In the case of isolation of tubular cells from an intact kidney, a catheter is inserted into a vein which either leaves or enters the kidney, collagenase solution is perfused through and tubular cells are released. In the case of a kidney biopsy, which results in a cut or exposed surface, a small catheter (or catheters) is inserted into vessels on the open or cut surface. Collagenase solution is perfused through the catheterized vessels, resulting in release of tubular cells. Once removed or isolated, the tubular cells are plated and maintained under conditions (e.g., on appropriate medium, at correct temperature, etc.) suitable for transfection.

Cells, e.g., tubular cells of the kidney or cardiac cells, containing the incorporated RNA interfering agents of the invention are grown to confluence in tissue culture vessels; removed from the culture vessel; and introduced into the body. This can be done surgically, for example. In this case, the tissue which is made up of transduced tubular cells capable of expressing the nucleotide sequence of interest is grafted or transplanted into the body. For example, it can be placed in the abdominal cavity in contact with/grafted onto the kidney or in close proximity to the kidney.

Alternatively, the transduced tubular cell-containing tissue can be attached to microcarrier beads, which are introduced (e.g., by injection) into the peritoneal space of the recipient Direct injection of genetically modified tubular cells into the kidney may also be possible.

If necessary, biochemical components needed for RNAi to occur can also be introduced into the cells, e.g., tubular cells of the kidney and cardiac cells.

Another aspect of the invention pertains to vectors, for example, recombinant expression vectors, containing a nucleic acid encoding an siRNA or shRNA of the present invention, e.g., apoptosis-related gene siRNA, e.g., Fas siRNA, or a cytokine siRNA, e.g., a proinflammatory siRNA such as a TNFα siRNA. As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, lentiviruses, adenoviruses and adeno-associated viruses), which serve equivalent functions. In a preferred embodiment, lentiviruses are used to deliver one or more siRNA molecule of the present invention to a cell.

In the preferred embodiment, the vectors contain siRNAs directed to human Fas and most preferably vectors comprising siRNAs directed against one or more sequences selected from the group consisting of hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3′ [SEQ ID NO: 17], and hFas siRNA 4 (beginning at nucleotide 1294) 5′-GGACATTACTAGTGACTCA-3′ [SEQ ID NO: 18].

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Furthermore, the RNA interfering agents may be delivered by way of a vector comprising a regulatory sequence to direct synthesis of the siRNAs of the invention at specific intervals, or over a specific time period. It will be appreciated by those skilled in the art that the design of the expression vector can depend on such factors as the choice of the target cell, the level of expression of siRNA desired, and the like.

The expression vectors of the invention can be introduced into target cells to thereby produce siRNA molecules of the present invention. In one embodiment, a DNA template, e.g., a DNA template encoding apoptosis-related genes, e.g. Fas, or a cytokine, e.g., proinflammatory cytokine, e.g., IL-1 or TNFα, may be ligated into an expression vector under the control of RNA polymerase III (Pol III), and delivered to a target cell. Pol III directs the synthesis of small, noncoding transcripts which 3′ ends are defined by termination within a stretch of 4-5 thymidines. Accordingly, DNA templates may be used to synthesize, in vivo, both sense and antisense strands of siRNAs which effect RNAi (Sui, et al. (2002) PNAS 99(8):5515).

The expression vectors of the invention may also be used to introduce shRNA into target cells. The useful expression vectors also be inducible vectors, such as tetracycline (see, e.g., Wang et al. Proc Natl Acad Sci U.S.A. 100: 5103-5106, 2003) or ecdysone inducible vectors (e.g., from Invitrogen) known to one skilled in the art.

As used herein, the term “target cell” is intended to refer to a cell, e.g., tubular cells of the kidney or cardiac cells, into which an siRNA molecule of the invention, including a recombinant expression vector encoding an siRNA of the invention, has been introduced. The terms “target cell” and “host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Preferably, a target cell is a mammalian cell, e.g., a human cell. In particularly preferred embodiments, it is a tubular cell of the kidney or a cardiac cell.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Preferred cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, B-galactosidase and the like may also be used to assess transfection efficiency. Cells which have been transfected with the siRNA molecules can then be identified by routine techniques such as immunofluorescence, phase contrast microscopy and fluorescence microscopy.

Depending on the abundance and the life-time (or turnover) of the targeted protein, a knock-down phenotype, e.g., a phenotype associated with siRNA inhibition of the target gene, e.g., apoptosis-related genes or cytokines, e.g., proinflammatory cytokines, e.g., IL-1 or TNFα, may become apparent after 1 to 3 days, or even later. In cases where no phenotype is observed, depletion of the protein may be observed by immunofluorescence or Western blotting. If the protein is still abundant after 3 days, cells can be split and transferred to a fresh 24-well plate for re-transfection.

If no knock-down of the targeted protein is observed, it may be desirable to analyze whether the target mRNA was effectively destroyed by the transfected siRNA duplex. Two days after transfection, total RNA can be prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable protein may exist in the cell. Multiple transfection in sufficiently long intervals may be necessary until the target protein is finally depleted to a point where a phenotype may become apparent.

RNA interfering agents of the instant invention also include, for example, small molecules which interfere with or inhibit expression of a target gene. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene. Assays to determine expression of the target gene, e.g., an apoptosis-related gene, or a cytokine, e.g., a proinflammatory cytokine, e.g., IL-1 or TNFα, and the activity or level of the protein encoded by the target gene, are known in the art. For example, reduced levels of target gene mRNA may be measured by in situ hybridization (Montgomery et al., (1998) Proc. Natl. Acad. Sci., USA 95:15502-15507) or Northern blot analysis (Ngo, et al. (1998)) Proc. Natl. Acad. Sci., USA 95:14687-14692).

Apoptosis-related gene polypeptide activity, e.g., Fas polypeptide activity, e.g., apoptosis, may also be assayed for by, for example, assays known in the art for cell death or apoptosis, such as, for example, transient transfection assays for cell death genes (as described in, for example, Miura M. et al. (2000) Methods in Enzymol. 322:480-92); assays that detect DNA cleavage in apoptotic cells (as described in, for example, Kauffman S. H. et al. (2000) Methods in Enzymol. 322:3-15); detection of apoptosis by annexin V labeling (as described in, for example, Bossy-Wetzel E. et al. (2000) Methods in Enzymol. 322:15-18); apoptotic nuclease assays (as described in, for example, Hughes F. M. (2000) Methods in Enzymol. 322:47-62); and analysis of apoptotic cells by flow and laser scanning cytometry (as described in, for example, Darzynkiewicz Z. et al. (2000) Methods in Enzymol. 322:18-39).

In another embodiment, the compositions of the invention are provided as a surface component of a lipid aggregate, such as a liposome, or are encapsulated by a liposome. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm. In one embodiment the invention features a lipid aggregate formulation of the compounds described herein, including phosphatidylcholine (of varying chain length; e.g., egg yolk phosphatidylcholine), cholesterol, a cationic lipid, and 1,2-distearoyl-sn-glycero3-phosphoethanolamine-polythyleneglycol-2000 (DSPE-PEG2000). The cationic lipid component of this lipid aggregate can be any cationic lipid known in the art such as dioleoyl 1,2,-diacyl trimethylammonium-propane (DOTAP). In another embodiment, polyethylene glycol (PEG) is covalently attached to the compositions of the present invention. The attached PEG can be any molecular weight but is preferably between 2000-50,000 daltons. In one embodiment for targeting macrophages for delivery of an RNA interfering agent, liposomes containing of phosphatidyl serine may be used since macrophage engulfment via the phosphatidyl serine receptor promotes an anti-inflammatory response by increasing TGF-β1 secretion (Huynh, M. L. et al. (2002) J. Cell Biol. 155, 649). Therefore, when the macrophages are successfully transfected, not only will the target genes be silenced, but the macrophage will also be induced to secrete anti-inflammatory cytokines.

In another embodiment, for delivery to a macrophage, a polyG tail, e.g. a 5-10 nucleotide tail, may be added to the 5′ end of the sense strand of the siRNA, which will enhance uptake via the macrophage scavenger receptor.

In another embodiment of the invention, the RNA interfering agents of the invention may be transported or conducted across biological membranes using carrier polymers which comprise, for example, contiguous, basic subunits, at a rate higher than the rate of transport of RNA interfering agents, e.g., siRNA molecules, which are not associated with carrier polymers. Combining a carrier polymer with an RNA interfering agents, e.g., an siRNA, with or without a cationic transfection agent, results in the association of the carrier polymer and the RNA interfering agent, e.g., siRNA. The carrier polymer may efficiently deliver the RNA interfering agent, e.g., siRNA, across biological membranes both in vitro and in vivo. Accordingly, the invention provides methods for delivery of an RNA interfering agent, e.g., an siRNA, across a biological membrane, e.g., a cellular membrane including, for example, a nuclear membrane, using a carrier polymer. The invention also provides compositions comprising an RNA interfering agent, e.g., an siRNA, in association with a carrier polymer.

The term “association” or “interaction” as used herein in reference to the association or interaction of an RNA interfering agent and a carrier polymer, refers to any association or interaction between an RNA interfering agent, e.g., an siRNA, with a carrier polymer, e.g., a peptide carrier, either by a direct linkage or an indirect linkage. An indirect linkage includes an association between a RNA interfering agent and a carrier polymer wherein said RNA interfering agent and said carrier polymer are attached via a linker moiety, e.g., they are not directly linked. Linker moieties include, but are not limited to, e.g., nucleic acid linker molecules, e.g., biodegradable nucleic acid linker molecules. A nucleic acid linker molecule may be, for example, a dimer, trimer, tetramer, or longer nucleic acid molecule, for example an oligonucleotide of about 2, 3, 4, 5, 6, 7, 8, 9, 10, 11, 12, 13, 14, 15, 16, 17, 18, 19, 20, 21, 22, 23, 24, 25, or more nucleotides in length.

A direct linkage includes any linkage wherein a linker moiety is not required. In one embodiment, a direct linkage includes a chemical or a physical interaction wherein the two moieties, e.g., the RNA interfering agent and the carrier polymer, interact such that they are attracted to each other. Examples of direct interactions include non-covalent interactions, hydrophobic/hydrophilic, ionic (e.g., electrostatic, coulombic attraction, ion-dipole, charge-transfer), Van der Waals, or hydrogen bonding, and chemical bonding, including the formation of a covalent bond. Accordingly, in one embodiment, the RNA interfering agent and the carrier polymer are not linked via a linker, e.g., they are directly linked. In a further embodiment, the RNA interfering agent and the carrier polymer are electrostatically associated with each other.

The term “polymer” as used herein, refers to a linear chain of two or more identical or non-identical subunits joined by covalent bonds. A peptide is an example of a polymer that can be composed of identical or non-identical amino acid subunits that are joined by peptide linkages.

The term “peptide” as used herein, refers to a compound made up of a single chain of D- or L-amino acids or a mixture of D- and L-amino acids joined by peptide bonds. Generally, peptides contain at least two amino acid residues and are less than about 50 amino acids in length.

The term “protein” as used herein, refers to a compound that is composed of linearly arranged amino acids linked by peptide bonds, but in contrast to peptides, has a well-defined conformation. Proteins, as opposed to peptides, generally consist of chains of 50 or more amino acids.

“Polypeptide” as used herein, refers to a polymer of at least two amino acid residues and which contains one or more peptide bonds. “Polypeptide” encompasses peptides and proteins, regardless of whether the polypeptide has a well-defined conformation.

In one embodiment, carrier polymers in accordance with the present invention contain short-length polymers of from about 6 to up to about 25 subunits. The carrier is effective to enhance the transport rate of the RNA interfering agent across the biological membrane relative to the transport rate of the biological agent alone. Although exemplified polymer compositions are peptides, the polymers may contain non-peptide backbones and/or subunits as discussed further below.

In an important aspect of the invention, the carrier polymers of the invention are particularly useful for transporting biologically active agents across cell or organelle membranes, when the RNA interfering agents are of the type that require trans-membrane transport to exert their biological effects. As a general matter, the carrier polymer used in the methods of the invention preferably includes a linear backbone of subunits. The backbone will usually comprise heteroatoms selected from carbon, nitrogen, oxygen, sulfur, and phosphorus, with the majority of backbone chain atoms usually consisting of carbon. Each subunit may contain a sidechain moiety that includes a terminal guanidino or amidino group.

Although the spacing between adjacent sidechain moieties will usually be consistent from subunit to subunit, the polymers used in the invention can also include variable spacing between sidechain moieties along the backbone.

The sidechain moieties extend away from the backbone such that the central guanidino or amidino carbon atom (to which the NH₂ groups are attached) is linked to the backbone by a sidechain linker that preferably contains at least 2 linker chain atoms, more preferably from 2 to 5 chain atoms, such that the central carbon atom is the third to sixth chain atom away from the backbone. The chain atoms are preferably provided as methylene carbon atoms, although one or more other atoms such as oxygen, sulfur, or nitrogen can also be present. Preferably, the sidechain linker between the backbone and the central carbon atom of the guanidino or amidino group is 4 chain atoms long, as exemplified by an arginine side chain.

The carrier polymer sequence of the invention can be flanked by one or more non-guanidino/non-amidino subunits, or a linker such as an aminocaproic acid group, which do not significantly affect the rate of membrane transport of the corresponding polymer-containing conjugate, such as glycine, alanine, and cysteine, for example. Also, any free amino terminal group can be capped with a blocking group, such as an acetyl or benzyl group, to prevent ubiquitination in vivo.

The carrier polymer of the invention can be prepared by straightforward synthetic schemes. Furthermore, the carrier polymers are usually substantially homogeneous in length and composition, so that they provide greater consistency and reproducibility in their effects than heterogenous mixtures.

According to an important aspect of the present invention, association of a single carrier polymer to an RNA interfering agent, e.g., an siRNA, is sufficient to substantially enhance the rate of uptake of an agent across biological membranes, even without requiring the presence of a large hydrophobic moiety in the conjugate. In fact, attaching a large hydrophobic moiety may significantly impede or prevent cross-membrane transport due to adhesion of the hydrophobic moiety to the lipid bilayer. Accordingly, the present invention includes carrier polymers that do not contain large hydrophobic moieties, such as lipid and fatty acid molecules.

In one embodiment, the transport polymer is composed of D- or L-amino acid residues. Use of naturally occurring L-amino acid residues in the transport polymers has the advantage that break-down products should be relatively non-toxic to the cell or organism. Preferred amino acid subunits are arginine (α-amino-delta.-guanidinovaleric acid) and α-amino-ε-amidinohexanoic acid (isosteric amidino analog). The guanidinium group in arginine has a pKa of about 12.5.

More generally, it is preferred that each polymer subunit contains a highly basic sidechain moiety which (i) has a pKa of greater than 11, more preferably 12.5 or greater, and (ii) contains, in its protonated state, at least two geminal amino groups (NH₂) which share a resonance-stabilized positive charge, which gives the moiety a bidentate character.

Other amino acids, such as α-amino-α-guanidinopropionic acid, α-amino-γ-guanidinobutyric acid, or α-amino-ε-guanidinocaproic acid can also be used (containing 2, 3 or 5 linker atoms, respectively, between the backbone chain and the central guanidinium carbon).

D-amino acids may also be used in the transport polymers. Compositions containing exclusively D-amino acids have the advantage of decreased enzymatic degradation. However, they may also remain largely intact within the target cell. Such stability is generally not problematic if the agent is biologically active when the polymer is still attached. For agents that are inactive in conjugate form, a linker that is cleavable at the site of action (e.g., by enzyme- or solvent-mediated cleavage within a cell) should be included to promote release of the agent in cells or organelles.

Any peptide, e.g., basic peptide, or fragment thereof, which is capable of crossing a biological membrane, either in vivo or in vitro, is included in the invention. These peptides can be synthesized by methods known to one of skill in the art. For example, several peptides have been identified which may be used as carrier peptides in the methods of the invention for transporting RNA interfering agents across biological membranes. These peptides include, for example, the homeodomain of antennapedia, a Drosophila transcription factor (Wang et al., (1995) PNAS USA., 92, 3318-3322); a fragment representing the hydrophobic region of the signal sequence of Kaposi fibroblast growth factor with or without NLS domain (Antopolsky et al. (1999) Bioconj. Chem., 10, 598-606); a signal peptide sequence of caiman crocodylus Ig(5) light chain (Chaloin et al. (1997) Biochem. Biophys. Res. Comm., 243, 601-608); a fusion sequence of HIV envelope glycoprotein gp4114, (Morris et al. (1997) Nucleic Acids Res., 25, 2730-2736); a transportan A-achimeric 27-mer consisting of N-terminal fragment of neuropeptide galanine and membrane interacting wasp venom peptide mastoporan (Lindgren et al., (2000), Bioconjugate Chem., 11, 619-626); a peptide derived from influenza virus hemagglutinin envelop glycoprotein (Bongartz et al., 1994, Nucleic Acids Res., 22, 468 1 4688); RGD peptide; and a peptide derived from the human immunodeficiency virus type-1 (“HIV-1”). Purified HIV-1 TAT protein is taken up from the surrounding medium by human cells growing in culture (A. D. Frankel and C. O. Pabo, (1988) Cell, 55, pp. 1189-93). TAT protein trans-activates certain HIV genes and is essential for viral replication. The full-length HIV-1 TAT protein has 86 amino acid residues. The HIV tat gene has two exons. TAT amino acids 1-72 are encoded by exon 1, and amino acids 73-86 are encoded by exon 2. The full-length TAT protein is characterized by a basic region which contains two lysines and six arginines (amino acids 47-57) and a cysteine-rich region which contains seven cysteine residues (amino acids 22-37). The basic region (i.e., amino acids 47-57) is thought to be important for nuclear localization. Ruben, S. et al., J. Virol. 63: 1-8 (1989); Hauber, J. et al., J. Virol. 63 1181-1187 (1989); Rudolph et al. (2003) 278(13):11411. The cysteine-rich region mediates the formation of metal-linked dimers in vitro (Frankel, A. D. et al., Science 240: 70-73 (1988); Frankel, A. D. et al., Proc. Natl. Acad. Sci. USA 85: 6297-6300 (1988)) and is essential for its activity as a transactivator (Garcia, J. A. et al., EMBO J. 7:3143 (1988); Sadaie, M. R. et al., J. Virol. 63: 1 (1989)). As in other regulatory proteins, the N-terminal region may be involved in protection against intracellular proteases (Bachmair, A. et al., Cell 56: 1019-1032 (1989).

In one embodiment of the invention, the basic peptide comprises amino acids 47-57 of the HIV-1 TAT peptide. In another embodiment, the basic peptide comprises amino acids 48-60 of the HIV-1 TAT peptide. In still another embodiment, the basic peptide comprises amino acids 49-57 of the HIV-1 TAT peptide. In yet another embodiment, the basic peptide comprises amino acids 49-57, 48-60, or 47-57 of the HIV-1 TAT peptide, does not comprise amino acids 22-36 of the HIV-1 TAT peptide, and does not comprise amino acids 73-86 of the HIV-1 TAT peptide. In still another embodiment, the specific peptides set forth in Table 1, below, or fragments thereof, may be used as carrier peptides in the methods and compositions of the invention.

TABLE 1

| | | |
|---|---|---|
| | | SEQ |
| | | ID |
| Peptide | Sequence | NO: |

| | | |
|---|---|---|
| HIV-1 TAT | RKKRRQRRR | 1 |
| (49-57) | | |
| HIV-1 TAT | GRKKRRQRRRT | 2 |
| 48-60 | PQ | |
| HIV-1 TAT | YGRKKRRQRRR | 3 |
| (47-57) | | |
| Kaposi | AAV ALL PAV | 4 |
| fibroblast | LLA LLA P + | |
| growth factor | VQR KRQ KLMP | |
| of caiman | MGL GLH LLV | 5 |
| crocodylus | LAA ALQ GA | |
| Ig(5) light | | |
| chain | | |
| HIV envelope | GAL FLG FLG | 6 |
| glycoprotein | AAG STM GA + | |
| gp41 | PKS KRK 5 (NLS | |
| | of the SV40) | |
| Drosophila | RQI KIW FQN | 7 |
| Antennapedia | RRM KWK K | |
| | amide | |
| RGD peptide | X-RGD-X | 8 |
| influenza | GLFEAIAGFIEN | 9 |
| virus | GWEGMIDGGGYC | |
| hemagglutinin | | |
| envelop | | |
| glycoprotein | | |
| transportan A | GWT LNS AGY | 10 |
| | LLG KIN LKA | |
| | LAA LAK KIL | |
| Pre-S-peptide | (S)DH QLN PAF | 11 |
| Somatostatin | (S)FC YWK TCT | 12 |
| (tyr-3- | | |
| octreotate) | | |

(s) optional Serine for coupling
italic = optional D isomer for stability

In yet another embodiment, an active thiol at the 5′ end of the sense strand may be coupled to a cysteine reside added to the C terminal end of a basic peptide for delivery into the cytosol (such as a fragment of tat or a fragment of the Drosophila Antennapedia peptide). Internalization via these peptides bypasses the endocytic pathway and therefore removes the danger of rapid degradation in the harsh lysosomal environment, and may reduce the concentration required for biological efficiency compared to free oligonucleotides.

Other arginine rich basic peptides are also included for use in the present invention. For example, a TAT analog comprising D-amino acid- and arginine-substituted TAT(47-60), RNA-binding peptides derived from virus proteins such as HIV-1 Rev, and flock house virus coat proteins, and the DNA binding sequences of leucine zipper proteins, such as cancer-related proteins c-Fos and c-Jun and the yeast transcription factor GCN4, all of which contain several arginine residues (see Futaki, et al (2001) J. Biol Chem 276(8):5836-5840 and Futaki, S. (2002) Int J. Pharm 245(1-2):1-7, which are incorporated herein by reference). In one embodiment, the arginine rich peptide contains about 4 to about 11 arginine residues. In another embodiment, the arginine residues are contiguous residues.

Subunits other than amino acids may also be selected for use in forming transport polymers. Such subunits may include, but are not limited to hydroxy amino acids, N-methyl-amino acids amino aldehydes, and the like, which result in polymers with reduced peptide bonds. Other subunit types can be used, depending on the nature of the selected backbone.

A variety of backbone types can be used to order and position the sidechain guanidino and/or amidino moieties, such as alkyl backbone moieties joined by thioethers or sulfonyl groups, hydroxy acid esters (equivalent to replacing amide linkages with ester linkages), replacing the alpha carbon with nitrogen to form an aza analog, alkyl backbone moieties joined by carbamate groups, polyethyleneimines (PEIs), and amino aldehydes, which result in polymers composed of secondary amines.

A more detailed backbone list includes N-substituted amide (CONR replaces CONH linkages), esters (CO₂), ketomethylene (COCH₂) reduced or methyleneamino (CH₂NH), thioamide (CSNH), phosphinate PO₂RCH₂), phosphonamidate and phosphonamidate ester (PO₂RNH), retropeptide (NHCO), transalkene (CR.dbd.CH), fluoroalkene (CF.dbd.CH), dimethylene (CH₂2CH₂), thioether (CH₂S), hydroxyethylene (CH(OH)CH₂), methyleneoxy (CH₂O), tetrazole (CN²4), retrothioamide (NHCS), retroreduced (NHCH₂), sulfonamido (SO₂NH), methylenesulfonamido (CHRSO₂NH), retrosulfonamide (NHSO₂), and peptoids (N-substituted glycines), and backbones with malonate and/or gem-diaminoalkyl subunits, for example, as reviewed by Fletcher et al. (1998) and detailed by references cited therein. Peptoid backbones (N-substituted glycines) can also be used. Many of the foregoing substitutions result in approximately isosteric polymer backbones relative to backbones formed from α-amino acids.

Polymers are constructed by any method known in the art. Exemplary peptide polymers can be produced synthetically, preferably using a peptide synthesizer (Applied Biosystems Model 433) or can be synthesized recombinantly by methods well known in the art.

N-methyl and hydroxy-amino acids can be substituted for conventional amino acids in solid phase peptide synthesis. However, production of polymers with reduced peptide bonds requires synthesis of the dimer of amino acids containing the reduced peptide bond. Such dimers are incorporated into polymers using standard solid phase synthesis procedures. Other synthesis procedures are well known in the art.

In one embodiment of the invention, an RNA interfering agent and the carrier polymer are combined together prior to contacting a biological membrane. Combining the RNA interfering agent and the carrier polymer results in an association of the agent and the carrier. In one embodiment, the RNA interfering agent and the carrier polymer are not indirectly linked together. Therefore, linkers are not required for the formation of the duplex. In another embodiment, the RNA interfering agent and the carrier polymer are bound together via electrostatic bonding.

It is known that depending upon the expression vector and transfection technique used, only a small fraction of cells may effectively uptake the siRNA molecule. In order to identify and select these cells, antibodies against a cellular target can be used to determine transfection efficiency through immunofluorescence. Preferred cellular targets include those which are present in the host cell type and whose expression is relatively constant, such as Lamin A/C. Alternatively, co-transfection with a plasmid containing a cellular marker, such as a CMV-driven EGFP-expression plasmid, luciferase, metalloprotease, BirA, β-galactosidase and the like may also be used to assess transfection efficiency. Cells which have been transfected with the siRNA molecules can then be identified by routine techniques such as immunofluorescence, phase contrast microscopy and fluorescence microscopy.

### Methods of Treatment:

The present invention provides for both prophylactic and therapeutic methods of treating a subject having or at risk for, or susceptible to, ischemia-reperfusion injury. As used herein, “treatment,” or “treating,” is defined as the application or administration of an interfering agent of the invention (e.g., an siRNA, e.g., an apoptosis-related gene siRNA or a cytokine siRNA, e.g., an IL-1 or TNFα siRNA) to a patient, or application or administration of a therapeutic agent to an isolated tissue or cell line from a patient, who has ischemia-reperfusion injury or inflammation, or symptoms thereof, with the purpose to cure, heal, alleviate, relieve, alter, remedy, ameliorate, improve or affect the ischemia-reperfusion injury or inflammation, or symptoms of the or ischemia-reperfusion injury.

In one preferred embodiment, the invention provides a method of treating ischemia reperfusion injury caused by Fas mediated apoptosis in a human subject in need thereof. In one embodiment, the method comprises administering human Fas-targeting siRNA to the blood supply vein of the affected organ in a human subject. The organ is preferably kidney or liver and the blood supply vein is preferably renal vein or hepatic vein, respectively.

With regard to both prophylactic and therapeutic methods of treatment, such treatments may be specifically tailored or modified, based on knowledge obtained from the field of pharmacogenomics. For example, the Fas gene of any subject human in need of Fas-targeting treatment may be sequenced and the Fas-targeting siRNA sequences adjusted to target the specific Fas mutations or polymorphisms in the subject individual.

In general, “pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to an RNA interfering agent (e.g., a patient's “siRNA response phenotype”, or “siRNA response genotype”). Thus, another aspect of the invention provides methods for tailoring an individual's prophylactic or therapeutic treatment with one or more RNA interfering agents, e.g., siRNAs or shRNAs, according to that individual's siRNA response genotype. Pharmacogenomics allows a clinician or physician to target prophylactic or therapeutic treatments to patients who will most benefit from the treatment and to avoid treatment of patients who will experience toxic drug-related side effects.

### Prophylactic Methods

In one aspect, the invention provides a method for preventing in a subject, an ischemia-reperfusion injury caused by or related to apoptosis-related gene activity, e.g., Fas activity, inflammation, or an immune response, or cytokine activity, e.g., inflammation, by administering to the subject one or more therapeutic agents, e.g., the RNA interfering agents as described herein (e.g., one or more siRNA, e.g., an apoptosis-related gene siRNA, e.g., a Fas siRNA, or a cytokine siRNA, e.g., an IL-1 or TNFα siRNA). Subjects at risk for an or ischemia-reperfusion injury, tissue injury, e.g., tubular cell of the kidney or cardiac cell injury caused by or related to apoptosis-related gene activity, e.g., Fas activity, inflammation, or an immune response, can be identified by, for example, any known risk factors for an or ischemia-reperfusion injury caused by or related to apoptosis-related gene activity, e.g., Fas activity, inflammation, or an immune response. Administration of a prophylactic agent can occur prior to the manifestation of symptoms characteristic of an ischemia-reperfusion injury caused by or related apoptosis-related gene activity, e.g., Fas activity, inflammation, or an immune response, such that the or ischemia-reperfusion injury, inflammation, or immune response are prevented or, alternatively, delayed in their progression. In the case of transplantation, the transplanted organ or tissue, e.g., kidney, heart, or lung, may be treated with the RNA interfering agents of the invention prior to transplantation or the RNA interfering agent may be administered after transplantation, via any known method or any method described herein.

In one preferred embodiment, the present invention provides a method of preventing ischemia reperfusion injury during an organ transfer from donor to the recipient comprising administering to either the donor or the recipient or both one or more Fas targeting siRNAs in pharmaceutically acceptable carrier, wherein inhibition of Fas expression in the target organ results in prevention or alleviation of ischemia reperfusion injury during the organ transplantation. The delivery is preferably performed by targeting one or more blood supply veins of the organ in question. In one preferred embodiment, the organ transplant is a kidney or liver transplant.

Any other mode of administration of the therapeutic agents of the invention, as described herein or as known in the art, including topical administration of the siRNAs of the instant invention, may be utilized for the prophylactic treatment of an or ischemia-reperfusion injury caused by or related to apoptosis-related gene activity, e.g., Fas activity, inflammation, or an immune response. Such topical administration may be performed, for example, using a spraying the siRNA on the organ to be transplanted or dipping or incubating the organ in a bath comprising the siRNA. A combination of topical administration with administration to one or more of the blood supply veins of the organ may also be used.

2. Therapeutic Methods

Another aspect of the invention pertains to methods of modulating gene expression or protein activity, e.g., apoptosis-related gene expression, e.g., Fas gene expression, or protein activity in order to treat ischemia-reperfusion injury. Accordingly, in an exemplary embodiment, the modulatory method of the invention involves contacting a cell expressing an apoptosis-related gene, e.g., Fas, or a cytokine, e.g., a proinflammatory cytokine, e.g., IL-1 or TNFα, with one or more RNA interfering agent (e.g., an siRNA, e.g., an apoptosis-related gene siRNA, e.g., Fas, or a cytokine siRNA) that is specific for the target gene, e.g., an apoptosis-related gene, e.g., Fas, or a cytokine, e.g., a proinflammatory cytokine, e.g. IL-1 or TNFα, such that expression of an apoptosis-related gene, e.g., Fas, or a cytokine, is modulated, e.g., an anti-apoptotic gene activity or cytokine activity, e.g., inflammation, is inhibited. These methods can be performed in vitro (e.g., by culturing the cell) or, alternatively, in vivo (e.g., by administering the agent to a subject).

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect, or analysis of appropriate patient samples (e.g., blood and/or tissues).

Generally, the amount needed is less than the amount needed in antisense treatment applications (see, e.g., Bertrand et al. Biochemical and Biophysical Research Communications 296: 1000-1004, 2002). Antisense therapy has been used in human treatment methods and a skilled artisan may seek additional guidance in dosaging, for example, from publications such as “Results of a Pilot Study Involving the Use of an Antisense Oligodeoxynucleotide Directed Against the Insulin-Like Growth Factor Type I Receptor in Malignant Astrocytomas” by David W. Andrews, et al. in J. Clin Oncol, April 15: 2189-2200, 2001.

The therapeutic compositions of the invention can also be administered to cells ex vivo, e.g., cells are removed from the subject, the compositions comprising the siRNAs or shRNAs of the invention are administered to the cells, and the cells are re-introduced into the subject. Vectors, e.g., gene therapy vectors, can be used to deliver the therapeutic agents to the cells. The cells may be re-introduced into the subject by, for example, intravenous injection.

The prophylactic or therapeutic pharmaceutical compositions of the invention can contain other pharmaceuticals, in conjunction with a vector according to the invention, when used to therapeutically treat or prevent an ischemia-reperfusion, and can also be administered in combination with other pharmaceuticals used to treat or prevent ischemia-reperfusion injury. For example, the prophylactic or therapeutic pharmaceutical compositions of the invention can also be used in combination with other pharmaceuticals which modulate the expression or activity of apoptosis-related genes, e.g., Fas, or cytokines, e.g., proinflammatory cytokines.

In the preferred embodiment, the RNA interfering agent is an siRNA targeting human Fas.

I. Pharmacogenomics

The RNA interfering agents as described herein (e.g., an siRNA, e.g., an apoptosis-related gene siRNA, e.g., a Fas or cytokine, e.g., proinflammatory cytokine, e.g., IL-1 or TNFα siRNA) can be administered to individuals to treat (prophylactically or therapeutically) ischemia-reperfusion injury. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer one or more therapeutic RNA interfering agents as described herein (e.g., an siRNA, e.g., an apoptosis-related gene siRNA, e.g., a Fas siRNA or cytokine, e.g., proinflammatory cytokine, e.g., IL-1 or TNFα siRNA) as well as tailoring the dosage and/or therapeutic regimen of treatment with an RNA interfering agent, e.g., an siRNA, e.g., an apoptosis-related gene siRNA, e.g., a Fas siRNA or a cytokine siRNA.

For example, in one embodiment, before administering the RNA interfering agent to an individual, the target sequence may be analyzed for any potential gene variations, such as polymorphisms or mutations, in the region against which the RNA interfering agent is targeted. For example, one may sequence the Fas genes. If one or more mutations or a polymorphisms is detected, the RNA interfering agent, such as siRNA, may be modified to target the specific mutant or polymorphic form of the target.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase (G6PD) deficiency is a common inherited enzymopa thy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug, such as siRNA, response. According to this method, if a gene that encodes a drug, such as siRNA, target is known, all common variants of that gene can be fairly easily identified in the population and it can be determined if having one version of the gene versus another is associated with a particular drug, such as siRNA, response.

As an illustrative embodiment, the activity of drug, such as siRNA, metabolizing enzymes is a major determinant of both the intensity and duration of drug, such as siRNA, action. The discovery of genetic polymorphisms of drug, such as siRNA, metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug, such as siRNA, response. For example, the gene expression of an animal dosed with a particular siRNA can give an indication whether gene pathways related to toxicity have been turned on.

Information generated from more than one of the above pharmacogenomics approaches can be used to determine appropriate dosage and treatment regimens for prophylactic or therapeutic treatment of an individual according to the methods of the present invention. This knowledge, when applied to dosing or drug selection, can avoid adverse reactions or therapeutic failure and thus enhance therapeutic or prophylactic efficiency when treating a subject with a therapeutic RNA interfering agent as described herein (e.g., an siRNA, e.g., an apoptosis-related gene siRNA, e.g., a Fas siRNA or a cytokine siRNA, e.g., proinflammatory cytokine, e.g., IL-1 or TNFα siRNA).

### Pharmaceutical Compositions

The RNA interfering agent, e.g., an siRNA of the invention can be incorporated into pharmaceutical compositions suitable for administration. Such compositions typically comprise the RNA interfering agent, e.g., an siRNA, such as an apoptosis-related gene siRNA, e.g., Fas siRNA or cytokine siRNA, and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

One preferred pharmaceutical composition according to the present invention comprises human Fas targeting siRNAs. Preferably, the human Fas targeting siRNAs are selected to target sequences selected from the group consisting of hFas siRNA 1 (beginning at nucleotide 457) 5′-GAGGAAGACTGTTACTACA-3′ [SEQ ID NO: 15], hFas siRNA 2 (beginning at nucleotide 667) 5′-TGATGAAGGACATGGCTTA-3′ [SEQ ID NO: 16], hFas siRNA 3 (beginning at nucleotide 1211) 5′-GAAGCGTATGACACATTGA-3′ [SEQ ID NO: 17], and hFas siRNA 4 (beginning at nucleotide 1294) 5′-GGACATTACTAGTGACTCA-3′ [SEQ ID NO: 18] or any combination thereof.

A pharmaceutical composition of the invention is formulated to be compatible with its intended route of administration. Generally, the compositions of the instant invention are introduced by any standard means, with or without stabilizers, buffers, and the like, to form a pharmaceutical composition. For use of a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

In one embodiment, the invention features the use of the compounds of the invention in a composition comprising surface-modified liposomes containing poly (ethylene glycol) lipids (PEG-modified, or long-circulating liposomes or stealth liposomes). In another embodiment, the invention features the use of compounds of the invention covalently attached to polyethylene glycol. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). The long-circulating compositions enhance the pharmacokinetics and pharmacodynamics of therapeutic compounds, such as DNA and RNA, particularly compared to conventional cationic liposomes which are known to accumulate in tissues of the MPS (Liu et al., J. Biol. Chem. 1995, 42, 2486424870; Choi et al., International PCT Publication No. WO 96/10391; Ansell et al., International PCT Publication No. WO 96/10390; Holland et al., International PCT Publication No. WO 96/10392). Long-circulating compositions are also likely to protect drugs from nuclease degradation to a greater extent compared to cationic liposomes, based on their ability to avoid accumulation in metabolically aggressive MPS tissues such as the liver and spleen.

Examples of routes of administration include parenteral, e.g., intravenous, intramuscular, intradermal, subcutaneous, oral (e.g., inhalation), transdermal (topical), transmucosal, vaginal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. The compounds can also be prepared in the form of suppositories (e.g. with conventional suppository bases such as cocoa butter and other glycerides) or retention enemas for rectal delivery.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to hepatocytes) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein.

Liposomal suspensions (including liposomes targeted to macrophages containing, for example, phosphatidylserine) can also be used as pharmaceutically acceptable carriers. These can be prepared according to methods known to those skilled in the art, for example, as described in U.S. Pat. No. 4,522,811 U.S. Pat. No. 5,643,599, the entire contents of which are incorporated herein. Alternatively, the therapeutic agents of the invention may be prepared by adding a poly-G tail to one or more ends of the siRNA for uptake into target cells. Moreover, siRNA may be fluoro-derivatized and delivered to the target cell as described by Capodici, et al. (2002) J. Immuno. 169(9):5196.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the siRNA in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules, oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of AN RNA interfering agent (i.e., an effective dosage) ranges from about 0.001 to 3000 mg/kg body weight, preferably about 0.01 to 2500 mg/kg body weight, more preferably about 0.1 to 2000 mg/kg body weight, and even more preferably about 1 to 1000 mg/kg, 2 to 900 mg/kg, 3 to 800 mg/kg, 4 to 700 mg/kg, or 5 to 600 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an RNA interfering agent can include a single treatment or, preferably, can include a series of treatments.

In a preferred example, a subject is treated with an RNA interfering agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of RNA interfering agent used for treatment may increase or decrease over the course of a particular treatment. Changes in dosage may result and become apparent from the results of diagnostic assays as described herein.

It is understood that appropriate doses of the RNA interfering agents, e.g., siRNAs or shRNAs, depend upon a number of factors within the ken of the ordinarily skilled physician, veterinarian, or researcher. The dose(s) of the agent will vary, for example, depending upon the identity, size, and condition of the subject or sample being treated, further depending upon the route by which the composition is to be administered, if applicable, and the effect which the practitioner desires the agent, e.g., an siRNA to have upon the target gene, e.g., an apoptosis-related gene, e.g., the Fas gene or a cytokine, e.g., proinflammatory cytokine, e.g., IL-1 or TNFα.

The RNA interfering agents, e.g., siRNAs of the invention can be inserted into vectors. These constructs can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the vector can include the RNA interfering agent, e.g., the siRNA vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

This invention is further illustrated by the following examples which should not be construed as limiting. The contents of all references, patents and published patent applications cited throughout this application, as well as the Appendix, and Figures, are incorporated herein by reference.

### EXAMPLE 1

### Fas Targeting siRNA Treatment Alleviates and Prevents Kidney Ischemia-Reperfusion Injury

Acute renal failure (ARF) often complicates critical illness and contributes to high morbidity and mortality at the intensive care units (ICU) (Liu, K. D. (2003) Crit. Care Med. 8(Suppl):S572-81). Furthermore, the management of ARF in the ICU patient is difficult (Heyman, S. N., et al. (2002) Curr. Opin. Crit. Care. 8(6):526-34). The most common cause of ARF is ischemic injury of tubular cells: acute tubular necrosis (ATN) due to decreased blood flow to the kidney (Prakash, J, et al. (2003) Ren. Fail. 25(2):225-33). Due to the osmotic gradient in the medulla, and the countercurrent concentration mechanism in the kidney, the most sensitive compartment to ischemia is the tubulointerstitium. In ischemia reperfusion injury of the kidney, organ failure is due to tubular cell death (Nogae, S., et al. (1998) J. Am. Soc. Nephrol. 9(4):620-31).

The clinical relevance of ATN is further aggravated by the limited ability of the adult mammalian kidney to regenerate; the lack of postnatal nephrogenesis in the human kidney. ATN due to ischemic injury leads to loss of epithelial cells eventually obstructing the tubular lumen by debris (Thadhani, R., et al. (1996) N. Engl. J. Med. 334: 1448-60). Thus, obstructive damage superimposes on the ischemic damage. The amount of material to be disposed of overloads the phagocytes, leading to deliberation of lysosomal enzymes and proinflammatory cellular metabolites injuring neighboring cells. In the end, necrosis extends the initial damage through amplification that often leads to life-threatening acute renal failure (Paolo, M., et al. (2003) J. Nephrol. 16: 186-195). Thus, reducing tubular epitheliai cell loss may be an effective therapeutic approach, to prevent such an amplification. Besides the integrity of the tubular basement membrane, which serves as a guide for reconstitution of a polarized epithelium, the key to successful repair after ATN is preserved blood supply to the tubulointerstitial compartment (Thadhani, R., et al. (1996) N. Engl. J. Med. 334: 1448-60). Thus, preserved integrity of the peritubular capillary system and the vasa recta may be crucial for survival after kidney ischemia.

Previous aims of gene-therapy to achieve sufficient gene expression in parenchymal organs included the use of hyperosmotic solutions, occlusion of the blood outflow, and hydrodynamic treatment (Zhang, G., et al. (1999) Hum. Gene Ther. 10:1735-7). In this later form, a large bolus, too large for the heart to handle is applied rapidly through the tail vein inducing fluid back-up in the system of the vena cava. Most of the applied material (DNA or siRNA) ends up in the liver. First, plasmid DNA was injected via this route, and the plasmid DNA expression could be detected primarily in hepatocytes (Song, E., et al. (2003) Nature Med. 9:347-351), but also in heart, lung, and kidney. The transgene expression in the organs other than the liver persisted longer, and was more stable (Maruyama, H., et al. (2002) J. Gene. Med. 4:333-41). Later, tail vein hydrodynamic treatment was also applied to deliver short interfering RNA (siRNA) treatment. One single injection of appropriate siRNA could achieve >90% downregulation in liver cells, and efficient downregulation in heart, lung, spleen, and kidney (Herweijer, H. and Wolff, J. A. (2003) Gene Therapy 10:453-458).

As the principle of hydrodynamic transduction seems to be fluid back-up in the system of vena cava, and increased hydrostatic pressure pushing the therapeutic molecules into the parenchymal interstitium where target cells take it up, target area of hydrodynamic treatment is the tubulointerstitium (Nagata, S, and Suda, T. (1995) Immunology Today 16:39-43). Thus, it is hypothesized that direct renal vein injection could achieve similar or higher efficiency with lower volumes used, and in the compartment of desire, the tubulointerstitium.

In addition to the tubulointerstitial compartment, endothelial integrity of the peritubular capillary network and the vasa recta might be crucial for oxygen supply to the tubulointerstitial compartment during recovery. Endothelial cells (EC) are normally resistant to apoptosis, despite constitutive FAS expression. This resistance is probably due to downstream regulation of FAS signaling by flice (caspase-8) inhibitory protein (FLIP). Ischemia reduces FLIP expression in endothelial cells, thus postischemic EC become sensitive to FAS mediated apoptosis (Sata, M., et al. (1998) J. Biol. Chem. 273, 33103-6; Scaffidi, C., et al. (1999) J. Biol. Chem. 274, 1541-8; Mogi, M., et al. (2001) Lab. Invest. 81: 177-184).

Based on the importance of FAS mediated apoptosis in kidney ischemia-reperfusion injury it was investigated whether temporary inhibition of FAS expression in the kidney by siRNA treatment could reduce kidney damage caused by ischemia-reperfusion. Inhibition of apoptosis during reperfusion may enhance postischemic kidney function recovery. Thus, preservation of the tubular epithelium, and the endothelium of the vasa recta, and the peritubular capillaries may reduce damage and enhance survival in a mouse kidney ischemia reperfusion model.

This example demonstrates that targeting of Fas with siRNA effectively reduces the translation of Fas and diminishes apoptosis and mortality in an in vivo model of kidney ischemia-reperfusion injury. It was found that injection of Fas siRNA decreased expression of Fas in the kidney, preserved the structure of tubular cells after 15, 30 and 45 minutes of ischemia in mice, and decreased mortality in mice subjected to kidney ischemia-reperfusion.

### Methods

Animals. Ten week-old male NMRI (Naval Medical Research Institute) mice weighing 27-32 gram (average: 30±1.3 gram) purchased from Toxi-Coop, Budapest, Hungary were used throughout the experiments. All procedures were performed in sterile conditions in accordance with guidelines set by the National Institutes of Health, The Institutional Animal Care and Use Committee of Semmerwies University, and the Hungarian law on animal care and protection.

Preparation of siRNAs. The siRNAs Chosen to Silence Fas Expression, have previously been shown to be effective (Song, Erwei, et al. (2003) Nature Med).

Deprotected and annealed siRNAs synthesized using 2′-O-ACE-RNA phosphoramidites (Dharmacon Research, Lafayette, Colo.), were dissolved in RNase-free PBS. The sense and anti-sense strands of siRNAs were as described in (8):

Fas sequence,

(SEQ ID NO: 13)

5⁵-P.GUGCAAGUGCAAACCAGACdTdT-3′ (sense),
and

(SEQ ID NO: 14)

5′-P.GUCUGGUUUGCACUUGCACdTdT-3′ (antisense);
and
GFP sequence,

[SEQ ID NO: 33]

5′-pGGCUACGUCCAGGAGCGCACC-3′ (sense)
and

[SEQ ID NO: 34]

5′-pUGCGCUCCUGGACGUAGCCUU-3′ (antisense).

Hydrodynamic treatment. The 2 side veins of the tail, or the penis vein were used for hydrodynamic injections. To dilate tail veins, the tail was immersed in warm water (50-55° C.), under ether narcosis for 5±1 seconds. A modified hydrodynamic treatment was used as described previously (Zhang, G., et al. (1999) Hum Gene Ther 10, 1735-7; Song, Erwei, et al. (2003) Nature Med). Briefly, 2.0 mg/kg-50 μg/25 g bodyweight siRNA/1 ml PBS was rapidly (1 ml within 10 seconds) pulse injected into the vein. Controls received saline (PBS) or GFP-siRNA pulse-injected under similar circumstances.

Application through the left renal vein. From a median laparotomy the left renal pedicle was visualized and the retroperitoneum was left intact to serve as tamponade after removal of the injection needle. Minimal preparation above the renal vessels was performed on the left side of the aorta: to insert an occlusion clip (BH31, Aesculap, Center Valley, Pa.). The aorta and the vena cava were clipped, and the renal vein was punctured with a 26 gauge needle, to inject 0.1 ml PBS containing siRNA or PBS (PBS=phosphate buffered saline). As average volume of the mice kidney is 0.1 ml. The needle was kept in place for 5 seconds, and than removed slowly, while applying compression to the renal vein with a piece of Gelaspon® (Chauvin Abkerpharm, Rudolstadt, Germany) held with forceps. The Gelaspon® was left in place thereafter. The aorta and vena cava clamp was removed immediately after the injection, having been maintained for total of at most about 10 seconds for each injection. With this method minimal bleeding was achieved.

Kidney ischemia-reperfusion. The possible deleterious effects of hydrodynamic treatment, or renal vein injection on kidney function, and an increased vulnerability of the kidney to subsequent ischemia-reperfusion injury were determined in pilot studies. No impairment of kidney function was detected, and survival after kidney ischemia-reperfusion was not influenced by these treatment modalities when vehicle (PBS) was used. These pilot studies also served to determine lethal (35 min) and sublethal (15 min) ischemic times in NMRI mice following renal vein and hydrodynamic treatment. As predominance of necrosis has been demonstrated over apoptosis in liver ischemia by increasing duration of ischemia (Sakai T, et al. (2003) Transplant Int. 16: 88-99), a relatively short ischemic time in the kidney, with presumably higher involvement of apoptotic cell death was chosen for the present experiments.

Kidney ischemia reperfusion was performed under standardized conditions: at 24±0.5° C. All general anesthetics so far tested markedly impair thermoregulatory control, increasing the range of temperatures not triggering protective responses (Sessler D L (1995). J Neurosurg Anesthesiol. 7(1):38-46.29) and body temperature importantly influences the outcome of ischemia reperfusion injury. Average intraabdominal temperature of the animals right after narcosis was 35±2° C., which was maintained during the whole operative period with a heating pad, controlled by the rectal temperature of the animal. The left renal pedicle was clamped for 15 or 35 minutes, and the right kidney was either left intact for control purposes (no renal vein injection, no ischemia), or removed for the survival experiments. For survival experiments, the animals were observed for several days after all surviving animals were free of signs of illness.

Experimental design. Renal vein injections from median laparotomy were performed on day 0. Animals were allowed 2 days to recover from surgery, and one single hydrodynamic venous treatment was performed on day 2. Following another 2 days of recovery, kidney ischemia-reperfusion was performed on day 4. By this time siRNA treatment should have reached maximal silencing effect. Animals were sacrificed 24 hours after ischemia for histologic and molecular biologic investigations, or observed for survival time. Two animals were harvested without kidney ischemia to determine silencing effectivity, and to compare systemic application alone (right kidney) with systemic plus renal vein treatment (left kidney).

| Pretreatment | Kidney ischemia | End-point | N = |
|---|---|---|---|
| FAS | 15 min | Harvest at 24 h | 5 |
| | 35 min | Survival | 4 |
| PBS | 15 min | Harvest at 24 h | 5 |
| | 35 min | Survival | 4 |
| FAS | None | Harvest for | 2 |
| PBS | None | silencing | 2 |

Functional measurements. Blood Urea Nitrogen (BUN) was measured on a Reflotron IV automate (Boehringer Mannheim, Germany) with a fast-test-strip, from 32 μL whole blood.

RNase protection assay. Total RNA was extracted from frozen kidney tissue using Trizol reagent (Molecular Research Center, Cincinnati, Ohio), and RNase protection assay (RPA) was performed using 15 μg of total RNA and the In-vitro Transcription Kit and mouse mAPO-3 multi-probe template set (BD Pharmingen, San Diego, Calif.) according to the manufacturer's instructions. Intensities of the protected bands were quantified by phosphor imaging (Fuji-BAS 1500; Fuji, Tokyo, Japan) based on the ratios of the investigated genes to GAPDH (internal control).

Real-Time PCR. Total RNA was isolated from whole kidneys by using TRIzol (Life Technologies, Gaithersburg, Md.). Primers for Fas and GAPDH were according to ref. 11. One-step real-time RT-PCR, using Sybr green reagent (Applied Bio-systems) for detection, was performed by using a Bio-Rad icycler. All reactions were done in a 50-μl reaction volume in triplicate, following the manufacturer's instructions. PCR parameters consisted of 30 min of reverse transcription at 48° C. and 10 min of Taq activation at 95° C., followed by 40 cycles of PCR at 95° C. for 20 sec, 55° C. for 30 sec, and 72° C. for 30 sec. Standard curves were generated for both Fas and GAPDH. Relative amounts of Fas mRNA were normalized to GAPDH mRNA. Specificity was verified by melting curve analysis and agarose gel electrophoresis.

Fas Immunohistochemistry. After deparaffinization and rehydration, paraffin sections of the kidneys were incubated with 3% hydrogenperoxide for 15 min to quench endogenous peroxidase activity. After microwaving for 20 ml, sections were blocked for 30 min in wash buffer containing 5% normal mouse serum. Sections were incubated for 1 h at room temperature with hamster anti-mouse Fas mAb (BD Pharmingen) diluted 1:100 in PBS. After washing with PBS, sections were incubated with biotinylated mouse anti-hamster Ig and then with streptavidin conjugated with horseradish peroxidase (LSAB detection kit, DAKO). After further washes in PBS, staining was developed with diaminobenzidine (DAB), and slides were lightly counter-stained with hematoxylin. Control slides were stained with hamster IgG replacing primary antibody. Fas immunostaining appears in all or none of the epithelial cells in individual renal tubules. The percentage of positive tubules in five consecutive fields of view (magnification, x200) was assessed in a blinded manner.

Terminal Deoxynucleotidyltransferase (TdT)-Mediated dUTP Nick End Labeling (TUNEL) Staining. Apoptosis of tubular epithelial cells was detected by in situ TUNEL assay (Roche Diagnostics) according to the supplier's instructions. Paraffin sections were deparaffinized in xylene and rehydrated before analysis. After endogenous peroxidase activity was quenched in 3% hydrogen peroxide for 20 min, sections were treated with proteinase K (20 mg/ml in 10 mM Tris.HCl, pH 7.6) at 37° C. for 30 min. before labeling with TdT and biotinylated dUTP in 100 mM potassium cacodylate/2 mM cobalt chloride/0.2 mM DTT, pH 7.2, at 37° C. for 60 min in a humidified chamber. TdT was omitted from control slides. Washed sections were incubated with peroxidase-labeled streptavidin for 30 min and then stained with diaminobenzidine, followed by counterstaining with hematoxylin. Approximately 1,000 tubular epithelial cells were counted by a blinded observer at high power (×400) to determine the percentage of TUNEL+ cells with apoptotic morphology.

Histologic Score. The mean was calculated from the blinded analysis of 50 cortical tubules with visible basement membrane on cross section by using a score of 0, no damage; 1, mild damage with rounding of epithelial cells and dilated tubular lumen; 2, severe damage with flattened epithelial cells, loss of nuclear staining, and dilated lumen; and 3, destroyed tubules with flat epithelial cells lacking nuclear staining.

Statistical Analysis. Statistical comparison was by two-sided Student's t test. Survival was analyzed by Kaplan-Meier test. Values are given as average ±standard deviation (SD). A p value of <0.05 was considered significant.

### Results

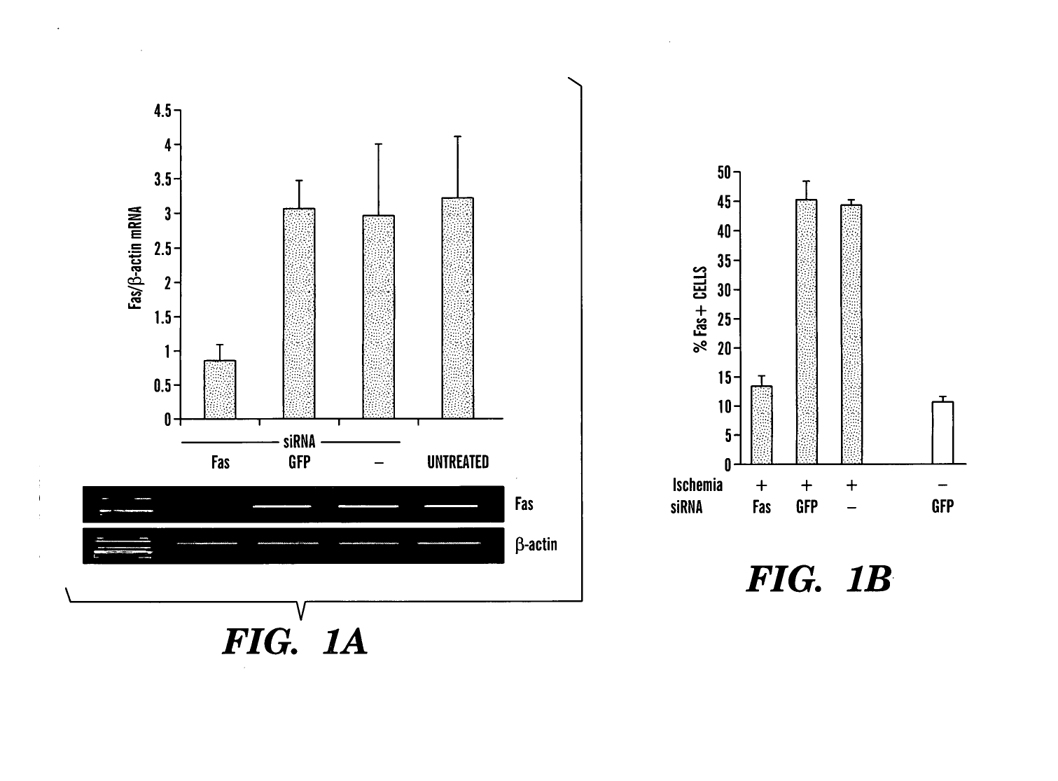

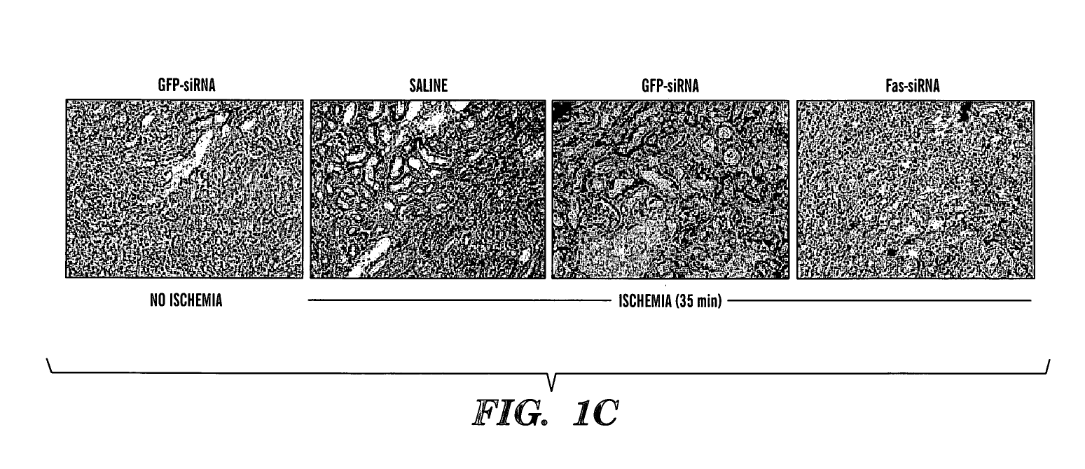

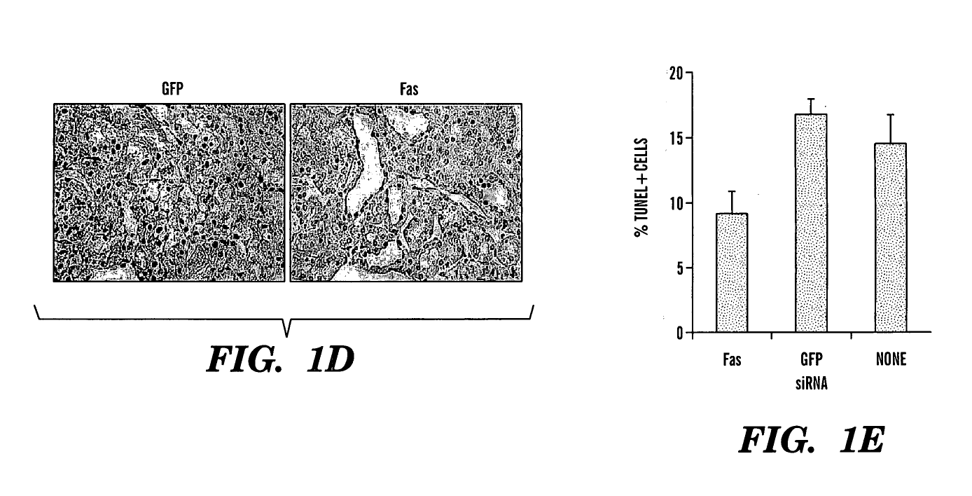

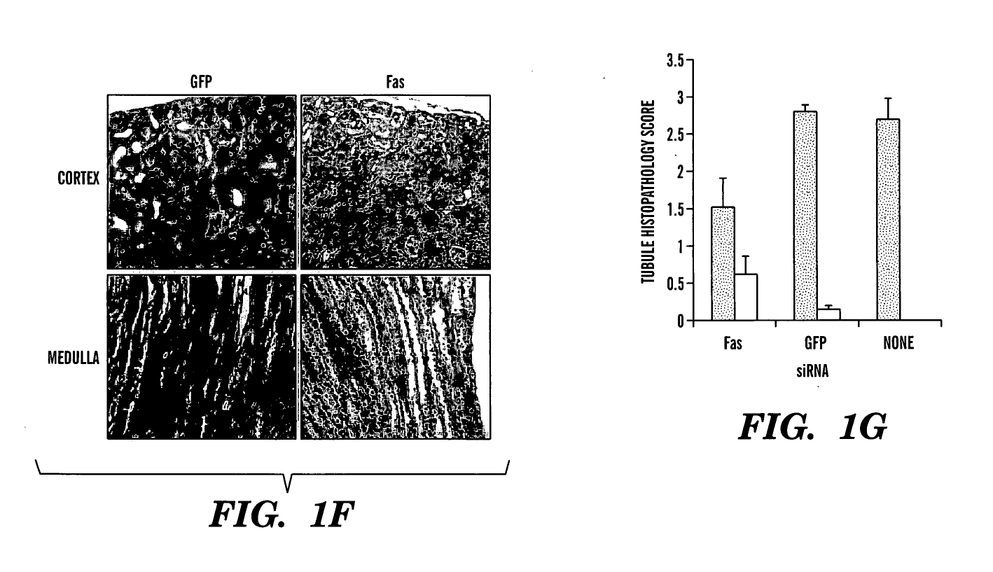

We first delivered synthetic siRNA duplexes (50 μg, 2.0-2.5 mg/kg) by a single hydrodynamic injection into the tail vein, using a Fas sequence that silenced effectively and specifically in the liver (8, 12). Twenty-four hours later, Fas mRNA in the kidney was reduced by 74±18% as determined by reverse transcriptase (RT)-PCR of whole kidney homogenates (FIG. 1a). Reduction in Fas mRNA was comparable to Fas silencing in the liver after three 50-μg hydrodynamic injections of the same siRNA (86% by RNase protection assay) (8).

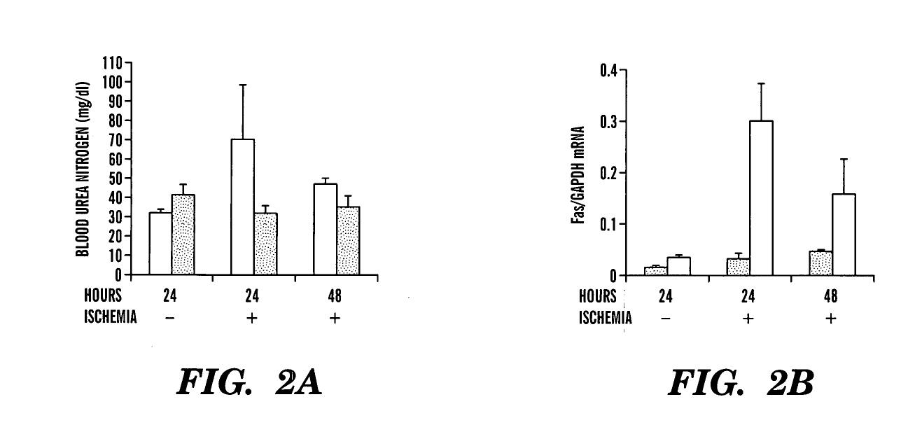

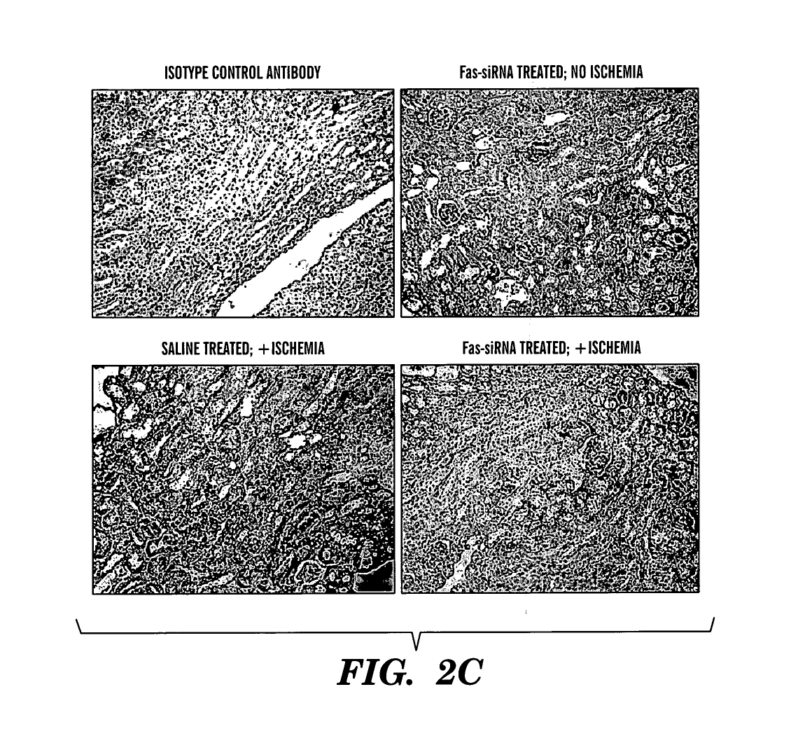

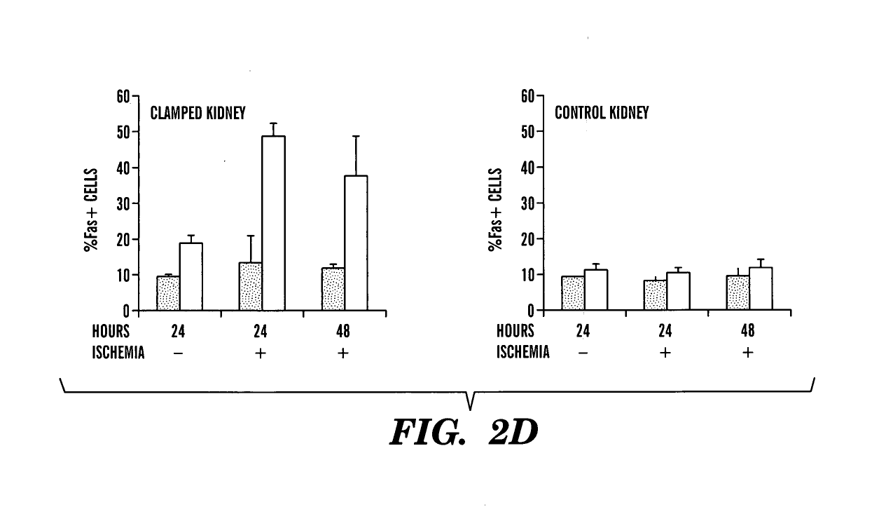

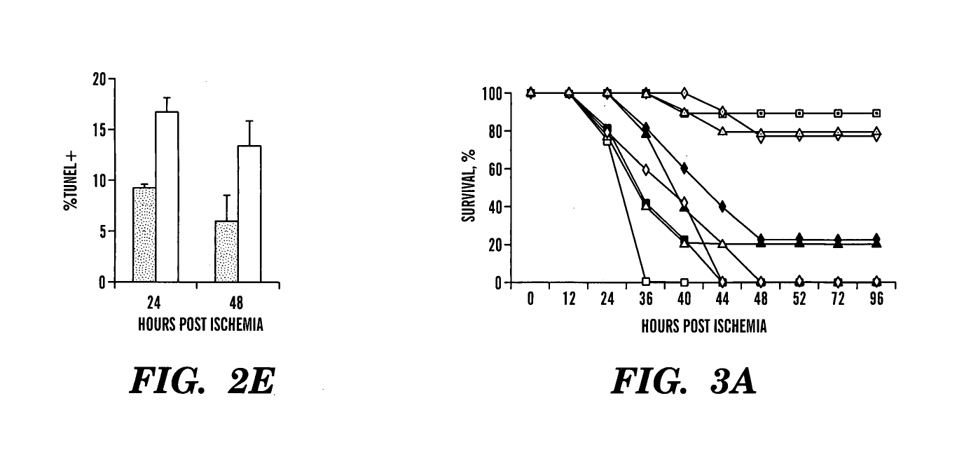

We next determined whether Fas siRNA injection could silence up-regulated Fas expression after ischemic damage. In pilot experiments in which the contralateral kidney was removed or clamped, 15 min of ischemia led to fatality in 16% (1 of 6) of mice and 30 min of ischemia killed 40-60% of mice, whereas 35-45 min of ischemia killed 80-100% of mice. The solitary fatal event after 15 min of ischemia may have been due to a cause other than acute tubular necrosis, because the typical increase in BUN after 15 min of ischemia was small and transient (see below and FIG. 2a).

These survival data suggest that the strain of mice used in these experiments is more sensitive to ischemic renal injury than are some inbred laboratory strains. In subsequent experiments we clamped the renal pedicle for either 15 or 35 min. Two days after a single hydrodynamic injection of 50 μg of Fas or GFP siRNA or saline, the left renal pedicle (artery and vein) was clamped for 35 min (leaving the right kidney intact), and mice were killed 1 day later for analysis of renal Fas expression and apoptosis. Fas silencing was also effective in the setting of ischemia. The ratio of Fas to GA PDH mRNA assayed by real-time PCR was 4 and 5 times lower in mice that received Fas siRNA compared with mice that received GFP siRNA (P<0.001) or hydrodynamic injection of saline (P<0.01), respectively (data not shown). The reduction in Fas mRNA up-regulation after ischemia in mice that received Fas siRNA was similar to the mRNA reduction in the nonischemic setting (FIG. 1a).

Histological sections prepared from both kidneys were stained for Fas protein expression and for TUNEL to detect apoptotic cells and were evaluated in a blinded manner for histological evidence of kidney damage (FIG. 1b-e). In the absence of ischemia after hydrodynamic injection of GFP siRNA, 10±1% of tubule epithelial cells stained for Fas. In clamped kidneys from GFP-siRNA- or saline-treated control mice, almost half of the tubule epithelial cells (45±3% and 44±1%, respectively) had detectable Fas protein. However, in Fas-siRNA-treated mice, only 13±2% of tubule cells stained for Fas. This percentage was statistically indistinguishable from Fas staining in control mice in which the renal pedicle was not clamped (P=0.13). The clamped kidneys from Fas-siRNA-treated mice also had significantly fewer apoptotic tubular epithelial cells (FIG. 1 d and e). Whereas 9±2% of renal tubule epithelial cells from Fas-siRNA-treated mice were TUNEL⁺, 17±1% of cells from mice that received GFP siRNA (P<0.001) and 14±2% of cells from saline-treated (P<0.01) were TUNEL⁺. Fas siRNA also protected the kidneys from ischemic damage, assessed by a blinded histopathology score that emphasized cortical tubular epithelial cell damage (FIG. 1 f and g). All control saline and GFP-siRNA-treated mice had extensive cortical tubular damage with massive tubular atrophy and cell loss with tubulointerstitial inflammatory cell infiltrates. Most surviving tubular epithelial cells had evidence of cytoplasmic swelling, and there was frequent nuclear chromatin condensation, indicative of apoptosis. In the medulla, tubular lumens were filled with hyaline material indicative of intense tubular cell loss in upper segments. In contrast, pretreatment with Fas siRNA prevented tubular epithelial cell loss and lessened inflammatory infiltration. The tubule histology score in the absence of ischemic insult in control siRNA-injected mice was <1 (on a scale that ranged from 0 to 3); it increased to 2.7±0.3 in the ischemic kidney of saline control mice and to 2.8±0.1 in GFP-siRNA-injected mice, but there was half as much damage (score 1.5±0.4) in Fas-siRNA-treated mouse kidneys (P<0.01 vs. saline, P<0.003 vs. GFP siRNA).

We next treated mice with a single hydrodynamic injection of Fas siRNA, as above, followed by a low-volume injection (50 μg in 0.1 ml) into the left renal vein 2 days later, and we induced subcritical ischemia 2 days after that by clamping the left renal pedicle for 15 min. If the right kidney was removed at the time of the renal injection, transient renal insufficiency developed in control mice (FIG. 2a). The BUN rose to 71±4 mg/dl the next day, compared with a normal value of 33±1 mg/dl without ischemia. Two days later the BUN had normalized. In mice treated with hydrodynamic and renal vein injections of Fas siRNA, the BUN remained normal (33±4 mg/dl 1 day and 36±6 mg/dl 2 days after clamping).

We next looked at how effectively Fas was silenced in the setting of 15 min of subcritical ischemia. In mice sham-treated with saline injections without clamping the renal pedicle, the Fas/GAPDH mRNA ratio by real-time PCR was 0.03±0.01, which was reduced to 0.015±0.01 in mice that received Fas siRNA (FIG. 2b). After subcritical ischemia, the ratio increased 10-fold to peak 1 day later at 0.30±0.07 and remained elevated at 0.16±0.07 2 days later.

However, in the mice that received Fas siRNA, the Fas/GAPDH mRNA ratio in the ischemic kidney hardly rose above that of the control mice not subjected to ischemia. The Fas/GAPDH ratio was 0.032±0.01 and 0.046±0.003 on days 1 and 2 after clamping (P<0.001 compared with control on day 1).

Moreover, Fas protein expression, assayed by counting the numbers of Fas-staining tubule cells by immunohistochemistry, was also substantially reduced in the challenged kidney (FIG. 2 c and d). In the Fas-siRNA-treated mice, 13±2% of tubule cells in the ischemic kidney became Fas⁺ at the peak response on day 1, whereas 49±4% of tubule cells stained for Fas in the ischemic control mouse kidney. Fas staining in the ischemic kidney of mice that received Fas siRNA was not significantly different from Fas staining in the nonischemic right kidney of control mice that received only saline injections. When the numbers of TUNEL⁺ apoptotic cells in the ischemic kidney were counted 1 and 2 days after clamping, there were about half as many TUNEL⁺ cells in the Fas-siRNA-treated mice as in the controls (FIG. 2e, P<0.002 day 1, P<0.05 day 2). Renal pathology determined by hematoxylin staining was also significantly reduced in the ischemic kidney with silenced Fas expression (tubule histopathology score, 1.4±0.5 in Fas-siRNA-treated animals vs. 2.2±0.3 in control mice, P<0.05).

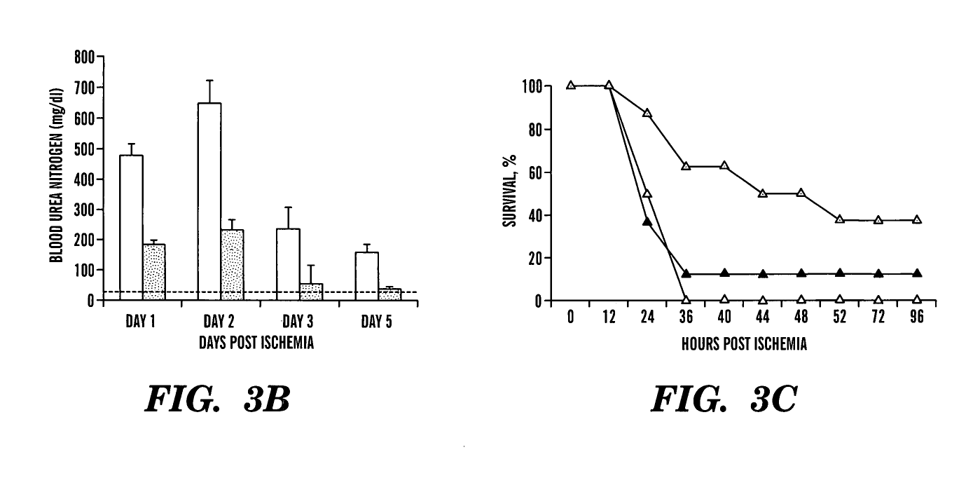

Because Fas expression in the kidney and tubular apoptosis and ischemic damage were suppressed, we next determined whether Fas siRNA could provide protection from critical ischemia in mice in which the left renal pedicle was clamped for 35 min and the contralateral kidney was removed. The right kidney was removed by median laparotomy 2 days before a single hydrodynamic injection of saline, GFP siRNA, or Fas siRNA. Two days later the renal pedicle of the remaining kidney was clamped for 35 min and the mice were observed (FIG. 3a). Four of five mice that received saline and all five mice that received GFP siRNA by hydrodynamic tail vein injection died of acute renal failure within 2 days. However, 8 of 10 mice injected with Fas siRNA survived (P<0.0001 vs. GFP siRNA, P<0.005 vs. saline). Kidney function, assessed by following BUN in surviving mice, was less perturbed in mice that received Fas siRNA than in those that received a hydrodynamic injection of saline (FIG. 3b. Whereas the peak BUN in surviving control mice was 646±77 mg/dl, it was a third of that (232±33 mg/dl, P<0.0001) in Fas-siRNA-treated mice, compared with a normal value of 33±1 mg/dl.

We also tested whether low-volume injection of siRNAs (100 μl) into the left renal vein (performed at the same time the right kidney was removed), which was well tolerated in the previous experiments, could enhance or substitute for hydrodynamic injection. The survival curves of mice treated with just renal vein infusion or with both hydrodynamic and renal vein injection were indistinguishable from those of mice that received a single hydrodynamic injection (FIG. 3a). Renal vein injection provided a significant survival advantage from critical ischemia-reperfusion injury (P<0.001 vs. GFP siRNA, P<0.01 vs. saline). Although hydrodynamic injection is unlikely to be possible in humans, catheterization of the renal vein is a preferable therapeutic option.

Although in some situations, such as preoperatively, it may be possible to anticipate ischemia-reperfusion injury, in most clinical situations ischemic damage arises without forewarning. We therefore evaluated survival when Fas siRNA was administered after the ischemic insult. Fas siRNA was injected in 100 μl into the renal vein during reperfusion 5 min after releasing the renal vessel clamp, when the kidney had recovered its red color. Postischemic renal vein injection protected three of eight mice from 35-min ischemia, whereas all eight GFP-siRNA-treated control mice and seven of eight saline-treated controls did not survive (P<0.01 vs. GFP siRNA, P 0.07 vs. saline). (FIG. 3c) In FIG. 3 a and c, all of the GFP-siRNA-treated mice died, whereas in each saline-treated group, one mouse survived. Although the differences between saline and GFP siRNA treatment in the experiments shown in FIG. 3 and elsewhere are not statistically significant, we cannot exclude subtle off-target effects induced by the GFP siRNA.

### Discussion

This study confirms the importance of Fas-mediated apoptosis in renal ischemia-reperfusion injury, because silencing Fas protected mice from lethal acute ischemic renal failure. The tissues of other organs, such as the heart or brain, might also be protected from ischemia-reperfusion injury by silencing Fas. Protection was provided not only by hydrodynamic injection but also by a single low-volume injection into the renal vein. How hydrodynamic injection works is not well understood. It is hypothesized that parenchymal cells are transduced when they are subjected to increased hydrostatic pressure induced by a sudden increase in intravascular volume. It is unlikely that this approach can be scaled up to human therapy. However, renal vein catheterization is feasible in humans and is likely to target the part of the kidney most vulnerable to ischemic damage, the tubulointerstitium.

In this study we chose an injection volume that roughly corresponds to the volume of a single mouse kidney (13). These injections may have created a transient localized increase in intravascular pressure within the kidney resulting in retrograde flow with siRNA transduction of tubule cells by a similar mechanism as for hydrodynamic injection, but without the risk of right-sided heart failure.

In a previous study we found that the Fas siRNA sequences used in this study specifically silenced Fas, but not other genes in the apoptotic pathway (8). However, recent in vitro studies have suggested that some duplex siRNA sequences have off-target effects and can induce an IFN response, particularly at high concentrations (14-16). Therefore, each new siRNA sequence should preferably be analyzed at least in the minimal gene expression profiling assay to screen for potential off-target effects. The preliminary in vitro studies in which cells were transfected with the Fas siRNA used in this study did not indicate any induction of IFN-inducible genes (data not shown). Without wishing to be bound by a theory, our data suggest that silencing Fas expression in renal tubular epithelial cells is the mechanism for protection by Fas siRNA injection. However, it is possible that indirect antiinflammatory effects of silencing Fas expression elsewhere contribute to the protective outcome.

Local injection of Fas siRNAs after ischemia provided some, but less complete, protection. Survival of Fas-siRNA-treated mice was significant when compared with GFP-siRNA-treated mice or with GFP-siRNA- and saline-treated control mice considered together (P<0.02), but not when compared with the saline-treated mice, although there was a trend toward protection. The half-life and delivery efficiency of duplex siRNAs in vivo can be improved by chemical modification (17, 18), which likely results in even more effective protection.

We thus conclude that postischemic protection is possible because Fas is up-regulated after ischemia, allowing a window of opportunity for therapeutic intervention.

Abbreviations used in this example: siRNA, small interfering RNA; BUN, blood urea nitrogen; TUNEL, terminal deoxynucleotidyltransferase-mediated dUTP nick end labeling.

All references cited herein and throughout the specification including the Example, are herein incorporated by reference in their entirety.

### REFERENCES

1. Nogae, S., Miyazaki, M., Kobayashi, N., Saito, T., Abe, K., Saito, H., Nakane, P. K, Nakanishi, Y. & Koji, T. (1998) J. Am. Soc. Nephrol. 9, 620-631. [Abstract]
2. Paschen, W. (2003) J. Cereb. Blood Flow Metab. 23, 773-779.[CrossRef][ISI][Medline]
3. Martin-Villalba, A., Hahne, M., Kleber, S., Vogel, J., Falk, W., Schenkel, J. & Krammer, P. H. (2001) Cell Death Differ. 8, 679-686. [CrossRef][ISI][Medline]
4. Castaneda, M. P., Swiatecka-Urban, A., Mitsnefes, M. M., Feuerstein, D., Kaskel, F. J., Tellis, V. & Devarajan, P. (2003) Transplantation 76, 50-54. [ISI][Medline]
5. Lee, P., Sata, M., Lefer, D. J., Factor, S. M., Walsh, K. & Kitsis, R. N. (2003) Am. J. Physiol. 284, H456-H463. [ISI]
6. Ortiz, A., Justo, P., Catalan, M. P., Sanz, A. B., Lorz, C. & Egido, J. (2002) Curr. Drug Targets Immune Endocr. Metabol. Disord. 2, 181-192. [Medline]
7. Miyazawa, S., Watanabe, H., Miyaji, C., Hotta, O. & Abo, T. (2002) J. Lab. Clin. Med. 139, 269-278. [CrossRef][ISI][Medline]
8. Song, E., Lee, S. K., Wang, J., Ince, N., Ouyang, N., Min, J., Chen, J., Shankar, P. & Lieberman, J. (2003) Nat. Med. 9, 347-351. [CrossRef][ISI][Medline]
9. McCaffrey, A. P., Meuse, L., Pham, T. T., Conklin, D. S., Hannon, G. J. & Kay, M. A. (2002) Nature 418, 38-39. [CrossRef][ISI][Medline]
10. Lewis, D. L., Hagstrom, J. E., Loomis, A. G., Wolff, J. A. & Herweijer, H. (2002) Nat. Genet. 32, 107-108. [CrossRef][ISI][Medline]
11. Terrazzino, S., Bauleo, A., Baldan, A. & Leon, A. (2002) J. Neuroimmunol. 124, 45-53. [CrossRef][ISI][Medline]
12. Zhang, G., Budker, V. & Wolff, J. A. (1999) Hum. Gene Ther. 10, 1735-1737. [CrossRef][ISI][Medline]
13. Maruyama, H., Higuchi, N., Nishikawa, Y., Hirahara, H., Iino, N., Kameda, S., Kawachi, H., Yaoita, E., Gejyo, F. & Miyazaki, J. (2002) Hum. Gene Ther. 13, 455-468. [CrossRef][ISI][Medline]
14. Jackson, A. L., Bartz, S. R., Schelter, J., Kobayashi, S. V., Burchard, J., Mao, M., Li, B., Cavet, G. & Linsley, P. S. (2003) Nat. Biotechnol. 21, 635-637. [CrossRef][ISI][Medline]
15. Sledz, C. A., Holko, M., de Veer, M. J., Silverman, R. H. & Williams, B. R. (2003) Nat. Cell Biol. 5, 834-839. [CrossRef][ISI][Medline]
16. Scacheri, P. C., Rozenblaft-Rosen, O., Caplen, N. J., Wolfsberg, T. G., Umayam, L., Lee, J. C., Hughes, C. M., Shanmugam, K. S., Bhattachaijee, A., Meyerson, M. & Collins, F. S. (2004) Proc. Natl. Acad. Sci. USA 101, 1892-1897. [Abstract/Free Full Text]
17. Hamada, M., Ohtsuka, T., Kawaida, R., Koizumi, M., Morita, K., Furukawa, H., Imanishi, T., Miyagishi, M. & Taira, K. (2002) Antiseise Nucleic Acid Drug Dev. 12, 301-309. [CrossRef][ISI][Medline]
18. Chiu, Y. L. & Rana, T. M. (2003) RNA 9, 1034-1048. [Abstract/Free Full Text]

1-25. (canceled)

26. A method of inhibiting Fas-protein regulated apoptosis in a cell comprising administering to the cell one or more short interfering RNAs (siRNA) which modulates Fas-protein encoding gene expression, thereby inhibiting apoptosis in the cell.

27. The method of claim 26, wherein the sequence of one or more siRNAs modulating human Fas protein expression comprises a nucleic acid selected from the group consisting of SEQ ID NO: 15, SEQ ID NO: 16, SEQ ID NO: 17 and SEQ ID NO: 18.

28. The method of claim 26, wherein said cell is a kidney cell.

29. The method of claim 28, wherein said kidney cell is a tubular cell.

30. The method of claim 26, wherein said cell is a cardiac cell.

31. A method of treating or preventing ischemia-reperfusion injury in a subject comprising administering to said subject a therapeutically or prophylactically effective amount of an RNA interfering agent targeting human Fas protein such that ischemia-reperfusion injury is treated or prevented.

32. The method of claim 31, wherein the subject is at risk for ischemia reperfusion injury in an organ, wherein the RNA interfering agent is one or more siRNAs targeting human Fas protein, wherein the one or more siRNAs and a pharmaceutically acceptable carrier is administered to a blood vessel of the organ, wherein the one or more siRNAs targeting human Fas protein inhibits Fas-protein expression in cells of the organ thereby inhibiting Fas-protein mediated apoptosis in the organ and preventing ischemia reperfusion injury in the organ.

33. The method of claim 31, wherein the sequence of one or more siRNAs targeting human Fas protein comprises a nucleic acid selected from the group consisting of SEQ ID NO: 15, SEQ ID NO: 16, SEQ ID NO: 17 and SEQ ID NO: 18.

34. The method of claim 31, further comprising a pharmaceutically acceptable carrier.

35. The method of claim 31, wherein ischemia-reperfusion injury affects any of the organs selected from the group consisting of kidney, heart, brain, liver, gut and lung.

36. The method of claim 31, wherein said subject is a human.

37. The method of claim 31, wherein said siRNA is administered intravenously.

38. The method of claim 37, wherein said siRNA is administered by repeated intravenous injection.

39. The method of claim 31, wherein the individual in need of is an organ transplant donor or organ transplant recipient.

40. A method of inhibiting Fas-protein mediated apoptosis in an organ in an individual in need thereof comprising administering to a blood vessel of an organ one or more siRNAs comprising a nucleic acid sequence targeting a sequence selected from the group consisting of SEQ ID NO: 15, SEQ ID NO: 16, SEQ ID NO: 17 and SEQ ID NO: 18 and a pharmaceutically acceptable carrier, wherein the siRNA inhibits Fas-protein expression in cells of the organ thereby inhibiting Fas-protein mediated apoptosis in the organ.

41. The method of claim 40, wherein the organ is kidney.

42. The method of claim 40, wherein the organ is heart.

43. The method of claim 40, wherein the individual in need of is either an organ donor or an organ recipient.
